(19) United States
(12) Patent Application Publication
(10) Pub. No.: US 2007/0219600 A1
(43) Pub. Date: Sep. 20, 2007

(54) DEVICES AND METHODS FOR TARGETED NASAL PHOTOTHERAPY

(76) Inventors: Michael Gertner, Menlo Park, CA (US); James R. Flom, San Mateo, CA (US); Peter L. Johnson, Mountain View, CA (US); Norbert H. Leclerc, Mountain View, CA (US); Erica Rogers, Redwood City, CA (US)

Correspondence Address: WILSON SONSINI GOODRICH & ROSATI, 650 PAGE MILL ROAD, PALO ALTO, CA, 94304-1050, US

(21) Appl. No.: 11/467,131

(22) Filed: Aug. 24, 2006

Related U.S. Application Data
(60) Provisional application No. 60/743,546, filed on Mar. 17, 2006.

(51) Int. Cl. A61N 5/06

(52) U.S. Cl. 607/88

(57) ABSTRACT

The invention is directed to therapy devices for providing therapeutic light to a target region of tissue within a nasal cavity. The devices are adapted to include a light source communicating with a distal end of the first insertion member, wherein light from the light source illuminates a target region of tissue within a nasal cavity posterior to a limen nasi of the nasal cavity, and further wherein the insertion member is configured to prevent illumination of a region of tissue anterior to the limen nasi. The devices are further adapted to be positioned within the nasal cavity by a user without observing the location of the distal tip.

### CROSS-REFERENCE

This application claims the benefit of U.S. Provisional Application No. 60/743,546, filed Mar. 17, 2006, which is incorporated herein by reference in its entirety.

### BACKGROUND OF THE INVENTION

1. Field of the Invention

The invention relates to devices and methods for delivering radiation to targeted regions of a nasal cavity, which includes the emission and propagation of energy in the form of rays or waves, including light, into the nasal cavity. The invention also relates to positioning a therapeutic device to deliver therapy posterior to the limen nasi using features specifically incorporated into the device and designed to provide an indication to the device operator as to the position of the device. The invention also relates to methods of manufacturing devices for delivering radiation to a nasal cavity including novel ultraviolet light sources such as LEDs.

2. Background of the Invention

The therapeutic use of light has been shown to be effective in the treatment of various medical conditions. For example, whole body exposure to ultraviolet (“UV”) light has been used for medical applications, such as the treatment of psoriasis and vitiligo. Ultraviolet lasers and lamps have also been designed to illuminate more localized regions of the skin for treatment of lesions and marks.

Infection of a patient takes many forms. Typically, acute bacterial infections are rather easily controlled using standard antibiotic therapies. Chronic infections, on the other hand, are often very difficult to control for several reasons: (1) the antimicrobial flora of chronically infected regions of the body often develop resistance to standard antibiotics due to multiple attempts to treat the flora with antimicrobial therapy; and (2) the microbes often form biofilms to protect themselves against the protective mechanisms of the patient.

Atopy refers to an inherited propensity to respond immunologically to many common, naturally occurring inhaled and ingested allergens with the continual production of IgE antibodies. Allergic rhinitis and asthma are the most common clinical manifestations of atopic disease affecting approximately 50 million people in the United States alone. There is a great deal of overlap among patients with atopic disease. For example, patients with atopic asthma have a greater likelihood of developing allergic rhinitis and dermatitis, and vice versa. Indeed, the pathophysiology for atopic diseases is generally the same whether or not the affected organ is the skin, the nose, the lungs, or the gastrointestinal tract.

Contact with an allergic particle (for example, pollen, cat dander, or food particle) reacts with an associated antibody on the mast cell, which leads to prompt mediator release and clinical symptoms. The IgE antibody response is perpetuated by T cells (antigen specific memory cells or other regulatory cells), which also have specificity for the allergens.

Kemeny, et al., in Intranasal Irradiation with the Xenon Chloride Ultraviolet B Laser Improves Allergic Rhinitis, 75 Journal of Photochemistry and Photobiology B: Biology 137-144 (2004) and Koreck, et al., in Rhinophototherapy: A New Therapeutic Tool for the Management of Allergic Rhinitis, Journal of Allergy and Clinical Immunology (March 2005), describe a treatment for allergic rhinitis using the same theory espoused for the efficacy of ultraviolet light in atopic dermatitis. Their placebo-controlled study showed the efficacy of ultraviolet therapy to treat allergic, or atopic, rhinitis over the course of an allergy season.

The United States Centers for Disease Control (CDC) estimates that each year, nearly 2 million people in the United States acquire an infection while in a hospital, resulting in 90,000 deaths. More than 70 percent of the bacteria that cause these infections are resistant to at least one of the antibiotics commonly used to treat them. Between 1979 and 1987, it is estimated that only 0.02 percent of pneumococcus strains infecting a large number of patients surveyed by the CDC were penicillin-resistant. As of 1994 that percent was estimated to have increased to 6.6 percent, and may currently approach 25%, by some estimates. Thus, as resistance increases, the importance of developing new treatment modalities increases.

A variety of devices are known for delivering light therapy. For example, U.S. Pat. No. 1,616,722 to Vernon for Kromayer Light Attachment; U.S. Pat. No. 1,800,277 to Boerstler for Method for Producing Therapeutic Rays; U.S. Pat. No. 2,227,422 to Boerstler for Applicator for Use in Treatment with Therapeutic Rays; U.S. Pat. No. 4,998,930 to Lundahl for Intracavity Laser Phototherapy Method; U.S. Pat. No. 5,146,917 to Wagnieres for Fiber-Optic Apparatus for the Photodynamic Treatment of Tumors; U.S. Pat. No. 5,292,346 to Ceravolo for Bactericidal Therapeutic Throat Gun; U.S. Pat. No. 5,683,436 to Mendes for Treatment of Rhinitus by Biostimulative Illumination; U.S. Pat. No. 6,663,659 to McDaniel for Method and Apparatus for the Photomodulation of Cells; and U.S. Pat. No. 6,890,346 to Ganz for Apparatus and Method for Debilitating or Killing Microorganisms within the Body. Additionally, U.S. Patent Publ. 2002/0029071 to Whitehurst for Therapeutic Light Source and Method; U.S. Patent Publ. 2004/0030368 to Kemeny for Phototherapeutical Method and System for the Treatment of Inflammatory and Hyperproliferative Disorders of the Nasal Mucosa; and U.S. Patent Publ. 2005/0107853 to Krespi for Control of Rhinosinusitus-Related, and Other Microorganisms in the Sino-Nasal Tract.

A variety of devices are known for delivering light and/or radiation. For example, PCT Publication WO 03/013653 to Kemeny et al. for Phototherapeutical Apparatus (see also, U.S. Patent Pub. US 2004/0030368 to Kemeny et al. for Phototherapeutical Method and System for the Treatment of Inflammatory and Hyperproliferaitve Disorders of the Nasal Mucosa); WO 2005/000389 to Fiset for Skin Tanning and Light Therapy Incorporating Light Emitting Diodes (see also, U.S. Patent Pub. 2004/0232339 to Lanoue for Hyperspectral Imaging Workstation Having Visible/Near-Infrared and Ultraviolet Image Sensors); U.S. Pat. No. 6,290,713 to Russell for Flexible Illuminators for Phototherapy; and U.S. Patent Pub. 2004/0176824 to Weckworth for Method and Apparatus for the Repigmentation of Human Skin.

### SUMMARY OF THE INVENTION

According to an aspect of the invention, a therapy device for providing therapeutic light to a target region of tissue within a nasal cavity is provided.

An aspect of the invention is directed to an optical therapy device for providing therapeutic light to a target region of tissue within a nasal cavity. Devices of the invention comprise a first insertion member; and a light source communicating with a distal end of the first insertion member, wherein light from the light source illuminates a target region of tissue within a nasal cavity posterior a limen nasi of the nasal cavity, and further wherein the distal end of the first insertion member is configured to prevent illumination of a region of tissue anterior to the limen nasi. In some embodiments, the insertion member can be configured to condition light exiting the distal end of the device, for example by scattering particles positioned at the distal end. In other embodiments, the insertion member can be adapted to further comprise an expandable component configured for positioning in a space between a nasal septum and the lateral wall of a nasal cavity. Expandable components can be, for example, a balloon and/or a device that is adapted to transmit light. In still other embodiments, the insertion member can further comprise a soft material. Suitable soft materials include, for example, elastomer, rubber, polymer, hydrogel, viscoelastic material, hydrophilic material, and hydrophobic material. In yet other embodiments, the insertion member can further be configured to provide a nasal anatomy information to an operator. Such configurations can include a sensor on the insertion member which provides anatomical measurements, or an imaging device on the insertion member which provides visual information. The devices of the invention can also further comprise a depth gauge adapted to set the depth of light delivery. In one embodiment, the insertion member can comprise a ruler. The device can also be comprised of components that, for example, engage with one another either before or during the process of delivering the device to the nasal cavity. For example, an insertion member can be provided that is positionable relative to a nasal bridge and then the therapy device is delivered relative to the component positioned relative to the nasal bridge. Additionally, a separable component can be provided that is adapted to be positioned between a lateral wall of a vestibule and the nasal septum and a second component which fits onto the optical therapy device and which is adapted to dock in the separable component. For example, the separable component can be configured to position the depth of the second component. In still other embodiments, the separable component is configured to determine the angle at which the second component directs the optical therapy device. In yet another embodiment of the device, the insertion member can be configured to provide one or more stackable elements configured to position the depth of the distal end. Additionally, a securement device can be provided to any embodiment that is adapted to engage a patient. The secure device is adapted to position the insertion member in the nasal cavity.

In still another embodiment of the invention, an optical therapy device for providing therapeutic light to a nasal cavity is provided. The devices comprises: one or more light sources; and an insertion member adapted to deliver therapeutic light from a light source to a first region of the nasal cavity while preventing light delivery to a second region, wherein the insertion member is positioned without visualization by an operator. A first region comprises respiratory epithelium and the second region is the squamous epithelium of the nasal vestibule. In some embodiments, the second region is one or more regions selected from the group consisting of a nasal vestibule, nasal septum, and a nasal valve. Additionally, the insertion member can further be configured to condition light exiting a light exit region. In still other embodiments, the interface portion can be further adapted to comprise an expandable component adapted to expand a space between a nasal septum and a lateral wall of the nasal cavity. Suitable expandable components include, for example, a balloon. In some embodiments, the insertion member is further adapted to partially block light from the light emitters and/or is comprised of a first part adapted and configured to expand a region between a nasal septum and a lateral wall of a nasal cavity and a second part having an optical therapy device adapted to dock in the first part.

In still another embodiment, an optical therapy device is provided that is adapted to provide therapeutic light to a nasal cavity. The devices comprise: an insertion member having a sensor adapted to provide nasal structure feedback to an operator; and one or more light sources adapted to illuminate the nasal cavity. In some embodiments, the feedback is visual information from an imaging element associated with the device. Additionally, the visual information can be obtained by way of a semiconductor imaging element.

The invention includes a method of applying phototherapy to a target region of a nasal cavity. The method comprises the steps of: (a) inserting a device having an insertion member into the nasal cavity; (b) positioning the insertion member at or near a target region; and (c) selectively delivering phototherapy to the target region of the nasal cavity. The insertion member used in the device can be further adapted to transmit target region information to an operator of the device regarding the target regions of the nasal cavity. Additionally, according to some embodiments of the method, the target region information is visual and/or anatomic measurements. In some embodiments of the method, the step of wedging open the limen nasi is included. Additionally, a step of adjusting the depth at which the distal tip of the insertion member emits light can be provided. Additionally, a step of adjusting a distance between a base of a first insertion member and a base of a second insertion member can also be performed. Further, in some embodiments, the step of inserting a device can further comprise the step of adjusting an angle between an axis of a first insertion member and an axis of a second insertion member. In still other embodiments, the step of inserting a device can further comprise the step of positioning an external depth gauge adjacent a target anatomical location. Additionally, in some embodiments, the step of inserting a device can further comprise the step of positioning a removable insertion guide over the insertion member prior to inserting the insertion member into the nasal cavity. In some embodiments, the step of inserting a device can further comprise the step of positioning a cap in the nasal cavity prior to a step of docking the insertion member into the cap. An additional step of securing the insertion member to a patient can also be performed in some of the embodiments. While still further, some methods can include the step of conditioning the phototherapy prior to delivery to the target region of the nasal cavity. A further step can include the step of measuring an anatomy of the nasal cavity.

An optical therapy device is also provided for delivering therapeutic light to a nasal cavity. In some embodiments, the therapy device comprises an insertion member configured to pivot relative to a connector positioned external a nasal cavity of a patient; and one or more light sources adapted to illuminate the nasal cavity. A second insertion member can be provided that is configured to pivot relative to the connector. Additionally, or in the alternative, a bridge can be provided between a first and second insertion member. The bridge can be configured to enable the first and second members to slideably engage. Additionally, the insertion member can pivot relative to the distal end.

### INCORPORATION BY REFERENCE

All publications, patents and patent applications mentioned in this specification are herein incorporated by reference to the same extent as if each individual publication, patent or patent application was specifically and individually indicated to be incorporated by reference.

### BRIEF DESCRIPTION OF THE DRAWINGS

A better understanding of the features and advantages of the present invention will be obtained by reference to the attached lab notebook pages and drawings wherein,

FIGS. 1A-E are an physiological views of the nasal passage illustrating the anatomy and cellular composition;

FIG. 2A is a cross-sectional view of a therapy device adaptable for use with an embodiment of the present invention; FIG. 2B is a cross-sectional view of a sheath of the therapy device of FIG. 2A;

FIG. 3 is a therapy system adaptable for use with an embodiment of the invention;

FIGS. 4A-B illustrate a distal tip arrangement for a therapy device of the present invention, such as the therapy device of FIG. 2;

FIG. 5 illustrates an alternative distal tip arrangement for a therapy device;

FIGS. 6A-B illustrates still another embodiment of a device suitable for delivering scattered light to a nasal cavity;

FIG. 7A illustrates a device adapted to measure a nasal cavity, or portion thereof, and illuminate a target region of the nasal cavity; FIG. 7B illustrates another device employing a custom depth gauge;

FIGS. 8A-B illustrate a measurement and therapy device configuration adapted to provide integrated measurement and therapy delivery;

FIGS. 9A-B illustrate a positioner adapted to provide control of the orientation and depth of the distal tip of the therapy device within a nasal cavity;

FIGS. 10A-C illustrate an adaptable therapy device that can be configured to provide an adjustable fit within the nasal cavity and securement to a patient;

FIGS. 11A-B illustrates alternate embodiments of a therapy device adapted to control the depth delivery of the treatment component;

FIGS. 12A-B illustrates an adaptably positionable therapy device positioned within a nasal cavity;

FIG. 13A-C illustrates distal configurations of a therapy device adapted to alter the shape or quality of a light beam therapeutically delivered within the nasal cavity;

FIG. 14 illustrates a distal tip of a device adapted to provide variable light delivery within the nasal cavity;

FIG. 15 illustrates an alternative distal tip configuration adapted for variable light delivery within the nasal cavity;

FIG. 16 illustrates a flow chart of a method for accessing a target nasal cavity for delivery of light therapy;

### DETAILED DESCRIPTION OF THE INVENTION

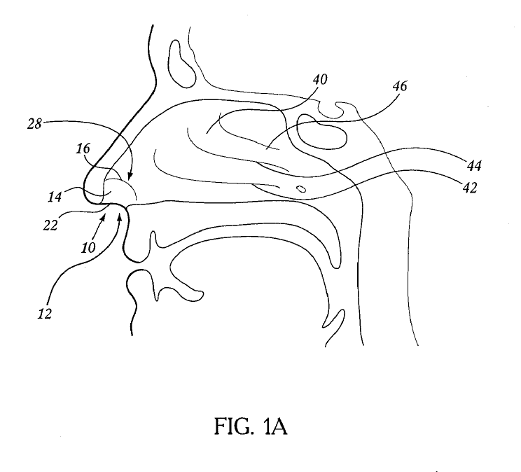

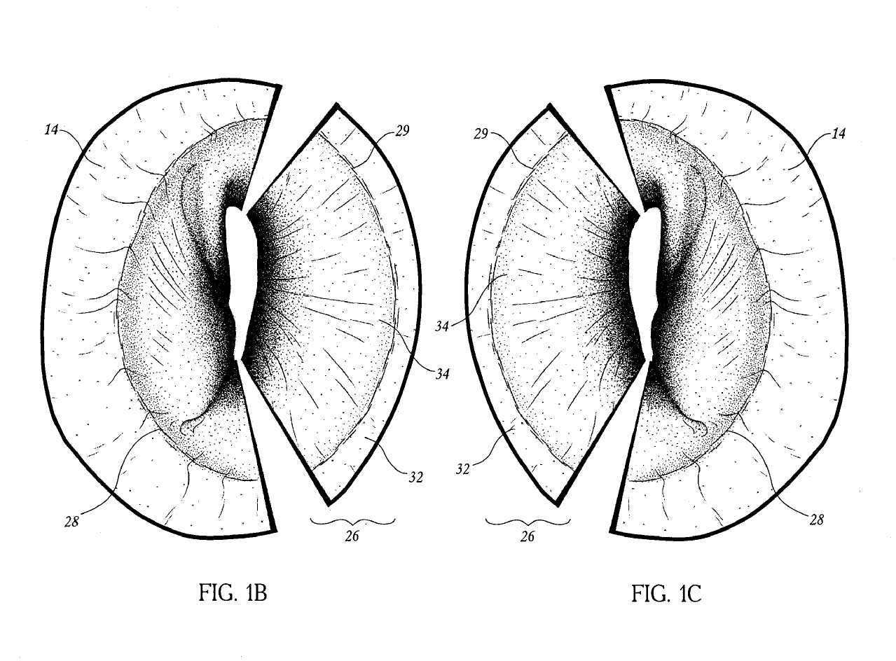

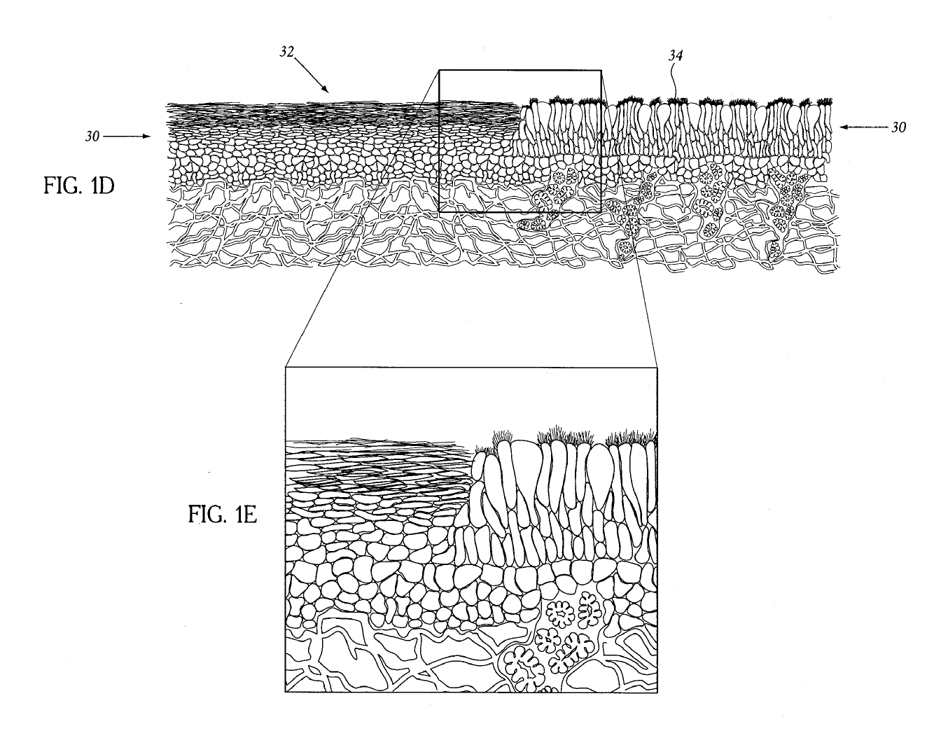

FIGS. 1A-E illustrates nasal anatomy and histology. FIG. 1A depicts a saggital section of the skull and face of a human. When the nasal cavity 10 is accessed through the anterior nares 12, the nasal vestibule (aka anterior vestibule) 14 is the first portion of the nasal cavity encountered. The limen nasi (aka vestibular limen) 16 is a ridge of skin, tissue, and mucosa that marks the transition between the squamous epithelium 32 and the respiratory epithelium 34 (shown in FIGS. 1D-E). The lateral wall 40 of the nasal cavity 10 is a complex structure containing three bony turbinates 42, 44, 46 with overlying mucosa consisting of stratified pseudocolumnar respiratory epithelia 34 as well as muscles (e.g. the nasalis muscle).

The view during a speculum exam (view looking into the nasal cavity without the assistance of an endoscope using a device which spreads apart the nasal valve 28) is depicted in FIGS. 1B-C. A physician encounters (in order of appearance): the squamous epithelium of the nasal vestibule, the limen nasi, the transition to respiratory epithelium, and the inferior turbinate 42. The middle and superior turbinates 44, 46 are encountered further back in the nasal cavity. On the septal side 26 (FIG. 1B-C), a transition zone 29 exists between squamous epithelium 32 and respiratory epithelium 34; however, the nasal valve tissue is limited to the lateral aspect of the nasal vestibule 14. The nasal septum 26 is a cartilaginous structure which is more rigid than the lateral aspect of the vestibule. Its transition zone 29 between squamous epithelium and respiratory mucosa occurs more superficially, approximately 1-3 mm inside the nasal cavity. The nasal bridge 22 is the representation of the septum 26 with the anterior nares 12 on either side when viewed from below. It appears as a vertical line of tissue, again when viewed from below.

Histologically, as illustrated in FIGS. 1D-E, the epithelial layer 30 of the anterior vestibule transitions from squamous epithelium 32 to respiratory epithelium 34 at a region termed the vestibular limen (aka limen nasi) 16 which is the ridge marking the boundary between the nasal cavity proper and the vestibule of the nose. This histologic transition is an area where tissues on either side of the transition zone respond differently to radiation; furthermore, each structure has its own sensory apparatus.

Kemeny et. al (U.S. Patent Publication US 2004/0030368) teach a method of nasal phototherapy with a patient interface. The interface is a simple rigid probe which does not allow the user to delineate between difference regions of the nasal cavity when the probe is applied to the nasal cavity. It has since been learned (see examples below) that when a patient interface such as that which is described in the Kemeny patent application and publications is used to treat the nasal cavity with ultraviolet light phototherapy, certain regions of the interior of the nasal cavity respond differently to the light. For example, as discussed above, the most anterior portion of the interior of the nose (the vestibule region where the squamous epithelium resides) undergoes a histologic transformation from respiratory epithelium to squamous epithelium. The squamous epithelium has been found to be more sensitive to ultraviolet light and mechanical irritation than the respiratory epithelium (see Examples below).

The patient interface described and taught by Kemeny exposes squamous epithelial cells of the nasal cavity to excessive amounts of ultraviolet light for several reasons: (1) the light is very close to this tissue; (2) the rim of tissue which makes up the vestibular limen draws the skin over the patient interface and tends to block its light and as a consequence, absorbs even more light; (3) the limen is exposed to mechanical trauma because as the skin draws over the patient interface, the limen physically blocks movement of the patient interface; (4) as the skin in this region is similar to squamous epithelium, it behaves as such and undergoes hyperproliferative and inflammatory reactions similar to a sunburn that one would see on the skin. This “sunburn” reaction is likely the cause of the “dryness” and pain encountered in previous clinical studies (Koreck et. al. “Rhinophototherapy: A New Therapeutic Tool for the Treatment of Allergic Rhinitis; J. All. Clin. Immunology: March 2005).

To overcome the above device and clinical limitations, it is desirable to utilize a patient interface which allows for partial or full control over the portion of the nasal cavity to where the light is applied. In some embodiments, the devices can be adapted to provide a tip arrangement, either removable or integrally formed, that facilitates measurement of the interior of the nasal cavity. In other embodiments, detection of a region of the nasal cavity, such as the limen nasi, is accomplished with inexpensive imaging equipment. Such visual feedback can be relatively crude and inexpensive (for example, a relatively coarse picture from a CCD or CMOS chip), simply providing an additional indication to position.

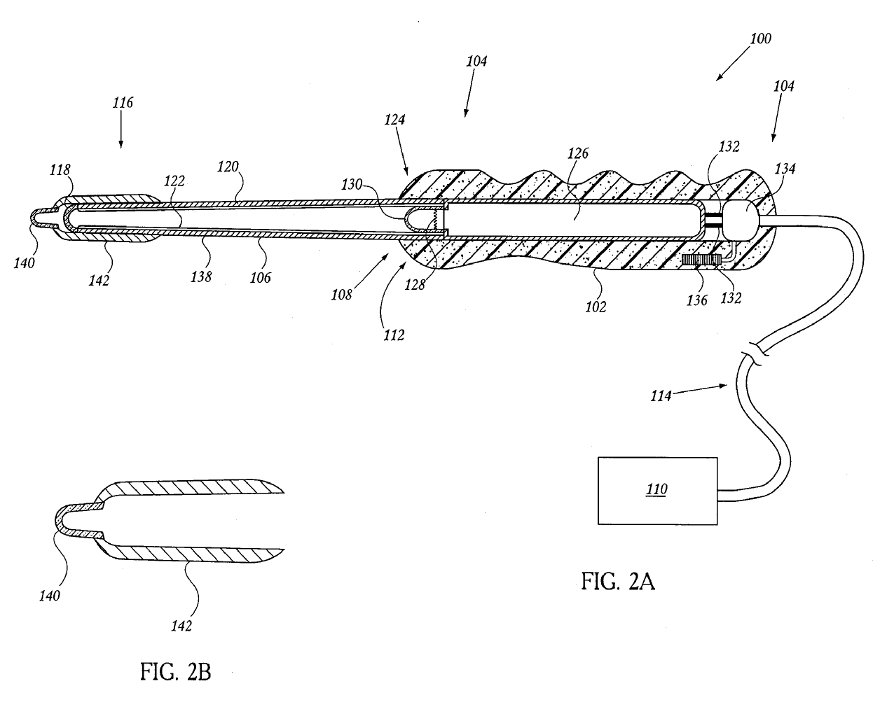

FIGS. 2A-B depict a therapy device 100 having a patient interface at the distal end 116. In one embodiment, the interface of therapy device 100 has a light delivery region 140, a handle or proximal portion 104, and a connector portion; in any of the embodiments, light can be generated within the nasal cavity or can be piped in through the connector portion of the interface at the distal end 116. In another embodiment, the therapy device 100 is coupled with a light source via a light communicating component, such as a lightguide. In this embodiment, the device may or may not have a handle 104.

The light entrance in some embodiments is delivered to the nasal cavity through a connector and, in other embodiments, the light is generated within the nasal cavity (see, e.g., U.S. patent application Ser. No. 11/152,946 to Gertner et al. for Therapy device). The distal portion of the patient interface 142 can, in one embodiment, wedge open the portion of the anterior nasal cavity which is not respiratory epithelium (the limen). A soft, compliant material either attached to or integral with the distal end of the patient interface facilitates opening of the nasal limen region. For purposes of illustration, the geometric profile of the therapy device 100 has been shown as having a rectangular profile (e.g. a length greater than a width). As will be appreciated by those skilled in the art, other profiles can be employed, either geometric or non-geometric (e.g., random) without departing from the scope of the invention. The various layers and elements of the therapy device 100 can be configured such that each provides a surface-to-surface contact with an adjacent element.

Turning now to the embodiments of the invention, a therapy device 100 for providing therapeutic light to a body cavity, such as the nasal cavity, in accordance with one embodiment of the present invention is illustrated in FIG. 2. The therapy device 100 in this embodiment generally includes a housing 102 or body which can be adapted to be hand-held, as illustrated; and a light source 126 positioned in or near the housing. An insertion member 106 is provided having a distal end configured to be inserted into a target nasal cavity to illuminate tissue within the nasal cavity from the light source when the distal end of the insertion member is positioned within the nasal cavity.

The housing can also generally refer to the therapy device 100 without the light source 126, housing 102, and/or without the power supply 110 or power cords 114 connected. The body of the therapy device in combination with the light source 126 can be held in one's hand or hands for an extended period of time (e.g., a therapeutic time) without undue effort or discomfort, due to the lightweight, portable design of the device.

In one embodiment, the insertion member 106 includes a tip 118 at the distal end of the insertion member 106. The tip 118 of the insertion member 106 is any of a variety of optically transparent or partially transparent structures. As will be appreciated, optically transparent components or materials can include components or materials that are transparent to wavelengths between about 200 nm and about 800 nm. In some cases, optically transparent can refer to more narrow ranges of transparency. For example, optically transparent to ultraviolet (UV) light can refer to transparency in the range from about 200 nm to 400 nm; while optically transparent to ultraviolet B (UVB) can refer to transparency in the range from about 280 nm to about 320 nm.

In one embodiment, the tip 118 includes a window, a diffusing lens, a focusing lens, a filter, or a combination of one or more of such tip types or other tip types which allow the spectral output to be conditioned. The lens can include, for example, a device that causes radiation or light to converge or diverge. The conditioner can be configured to modify the spectral output or the geometric illumination pattern of the device. In one embodiment, to provide a desired output spectrum, three types of tips can be used in series within the insertion member 106. For example, in one embodiment, a lens is used to diffuse (e.g., refract) certain wavelengths while filtering (e.g., transmitting certain wavelengths and absorbing others) certain wavelengths, and serving as a window (e.g., transmitting) certain wavelengths. In another embodiment, the light from the insertion member 106 is transferred through tip 118 through a series of internal reflections. In one embodiment, the tip 118 is made at least in part from a different material than that of the insertion member 106. The tip 118 of the insertion member 106 may be shaped or designed to disperse light as it exits the reflecting insertion member 106 and is transmitted to a patient.

The insertion member 106 can be configured as a reflecting tube and can be manufactured from any of a variety of materials, including plastic, stainless steel, nickel titanium, glass, quartz, aluminum, rubber, lucite, or any other suitable material known to those of skill in the art that may be adapted to be place inside of a patient's nasal cavity. In some embodiments, the material of the tube is chosen to reflect certain wavelengths and/or absorb others. In some embodiments, the tube is configured to yield near or total internal reflection.

Additionally, the insertion member 106 can be configured such that it is hollow. Where the insertion member 106 is hollow, the inside wall 120 of the insertion member 106 can be adapted to at least partially reflect light of a selected wavelength. Thus, the inside wall 120 may include a reflecting layer 122 applied over its entire surface although in other embodiments the inside wall 120 does not include a reflecting layer 122. In one embodiment, the reflective layer 122 includes a coating of a reflecting material such as, for example, aluminum, silica carbide, or other suitably reflective material.

The proximal end 108 of the insertion member 106 is coupled to the distal end 112 of the hand piece 102 by any of a variety of couplings 124 well known to those of skill in the art. For example, a suitable coupling 124 includes a press-fit connection, a threaded connection, a weld, a quick-connect, a screw, an adhesive, or any other suitable coupling as is known to those of skill in the art. Coupling 124 includes mechanical, optical, and electrical couplings, as well as combinations thereof. In one embodiment, the coupling 124 is releasable so that the insertion member 106 may be decoupled or removed from the hand piece 102. Such coupling 124 may also be made from a disposable material. In another embodiment, the insertion member 106 is permanently attached to the hand piece 102. In such case, the coupling 124 is a permanent connection.

In one embodiment, the hand piece 102 of the body includes a light source 126. The light source may be any of a variety of high, low, or medium pressure light emitting devices such as for example, a bulb, an emitter, a light emitting diode (LED), a xenon lamp, a quartz halogen lamp, a standard halogen lamp, a tungsten filament lamp, or a double bore capillary tube, such as a mercury vapor lamp with or without a phosphor coating. The particular light source selected will vary depending upon the desired optical spectrum and the desired clinical results, as will be described in greater detail below. Although the light source 126 of FIG. 2 is shown in the hand piece 102, the light source 126 can be placed anywhere on, in, or along the therapy device 100, including on or in the distal end of the insertion member. In some of the embodiments discussed below, multiple light sources are adapted for delivery by the therapy device 100, some of which may reside in the hand piece 102, some of which may reside on or in the insertion member 106, some of which may reside on or in the tip 118, and some of which may reside elsewhere in the therapy device 100 and communicate with the distal end 116 of the insertion member 106.

The light source 126 can be configured to include a phosphor-coated, low pressure mercury vapor lamp. In a related embodiment, the phosphor is placed distal to the mercury vapor lamp; for example, the phosphor is coated onto the reflecting layer 122 or is incorporated into the tip 118. Optical emitter 128 illuminates the light emitting portion of the light source 126. When light source 126 is a mercury vapor lamp, optical emitter 128 can be an inner capillary tube where the mercury plasma emits photons. Optical emitter 128 can also comprise a filament. Such filaments may be used when light source 126 is an incandescent or halogen lamp. Leads 132 extending from the light source 126, electrically couple the light source 126 with a control circuit 134. In one embodiment, the control circuit 134 is in electrical communication with a controller 136 and with power supply 110 via the power coupling 114.

As will be appreciated by those skilled in the art, it may be desirable to control variables or control parameters associated with the output of the therapy device 100. Examples of such variables include power, timing, frequency, duty cycle, spectral output, and illumination pattern. In one embodiment, the control circuit 134 controls the delivery of power from the power supply 110 to the light source 126 according to the activation or status of the controller 136. For example, in one embodiment, the control circuit 134 includes a relay, or a transistor, and the controller 136 includes a button, or a switch. When the button or switch of the controller 136 is pressed or activated, power from the power supply 110 is able to flow through the control circuit 134 to the light source 126.

The variables can be controlled in response to, for example, at least one photoreflectance parameter, which, for example, may be measured or obtained at the distal end 116 of the therapy device 100. Other variables or control parameters include desired dosage, or a previous dosage. In some embodiments, the patient or treating physician can adjust the treatment time based on the prior history with the therapy device 100. In some embodiments, controller mechanisms, which can be integral to the therapy device 100, allow for control over dosage and illumination. In other embodiments, the controller tracks the total dose delivered to a patient over a period of time (e.g., seconds, minutes, hours, days, months, years) and can prohibit the device from delivering additional doses after the preset dosage is achieved.

Although the control circuit 134 is illustrated within the hand piece 102 of the therapy device 100, in another embodiment, the control circuit 134 is located within the power supply 110. Other configurations can be employed without departing from the scope of the invention. In such embodiments, the controller 136 communicates with the control circuit 134 through the power coupling 114. Control data, commands, or other information may be provided between the power supply 110 and the hand piece 102 as desired. In one embodiment, control circuit 134 stores information and data, and can be coupled with another computer or computing device.

In one embodiment, power from the power supply 110 flows to the control circuit 134 of the hand piece 102 through a power coupling 114. The power coupling 114 may be any of a variety of devices known to those of skill in the art suitable for providing electrical communication between two components. For example, in one embodiment, the power coupling 114 includes a wire, a radio frequency (RF) link, or a cable.

In another embodiment, the light source 126 is housed separately from the hand piece 102 and communicates directly with the insertion member 106 and/or hand piece 102 via a light guide or light communicating member. In this embodiment, the insertion member 106 may or may not be housed by a hand piece 102. The hand piece 102 may or may not be comprised of a controller 136, control circuit 134 and/or leads 132.

The light source 126 is generally adapted to emit light with at least some wavelengths in the ultraviolet spectrum, including the portions of the ultraviolet spectrum known to those of skill in the art as the UVA (or UV-A), UVA₁, UVA₂, the UVB (or UV-B) and the UVC (or UV-C) portions. In another embodiment of the current invention, light source 126 emits light in the visible spectrum in combination with ultraviolet light or by itself. Finally, in yet another embodiment, the light source 126 emits light within the infrared spectrum, in combination with white light and/or ultraviolet light, or by itself. It is generally understood that these spectra have the following definitions: 100-280 nm for UVC, 280-320 nm for UVB, 320-400 nm for UVA, 400-800 nm for visible, and 800-11,000 nm for infrared.

Light source 126 may be adapted to emit light in more than one spectrum simultaneously (with various phosphors, for example) or a multiplicity of light sources may be provided to generate more than one spectrum simultaneously. Light emission at these spectra can be characterized as broad- or narrow-band emission. In one embodiment, narrow-band is over a band gap of about 10-20 nm and broad-band is over a band gap of about 20-50 nm. For example, in one embodiment, the light source 126 emits light having a narrow-band wavelength of approximately 308 nm within the UVB portion of the UV spectrum. In another embodiment, the light source 126 emits light having a wavelength below approximately 300 nm. In other embodiments, the light source 126 emits light having a wavelength between about 254 nm and about 313 nm.

The spectrum delivered can be continuous or intermittent. Continuous (or substantially continuous) refers to generally smooth uniform optical output. Intermittent refers to one or more pauses or lapses in optical output. In other embodiments, the light source 126 emits light in any two of the foregoing spectra and/or spectra portions in a continuous manner. In another embodiment, the light source 126 emits light in any two of the foregoing spectra and/or spectra portions in an intermittent manner. In still other embodiments, some portions of the spectra are continuous and others intermittent.

The therapy device 100 can be configured to include more than one light source 126, where each light source 126 has an output centered at a different wavelength. Each light source 126 can have an output that can be characterized as broad-band, narrow-band, or substantially single band. All light sources 126 can be the same characterization, or may have one or more different characterizations. For example, in one embodiment, the therapy device 100 includes three light sources 126: one that emits light in the UVA region of the UV spectrum, one that emits light in the UVB region of the UV spectrum, and one that emits light in the visible region of the optical spectrum.

The light sources may each emit light at a different energy or optical power level, or at the same level. The therapy device 100 may be configured to provide light from three light sources 126, each having a different relative output energy and/or relative energy density level (e.g., fluence).

Optical energy densities are generally derived from a power density applied over a period of time. Various energy densities are desired depending on the disorder being treated and may also depend on the light source used to achieve the optical output. For example, in some embodiments, the energy densities are achieved over a period of time of about 0.5 to 3 minutes, or from about 0.1 to 1 minute, or from about 2 to 5 minutes. In some embodiments, for example, when a laser light source is used, the time for achieving these energy density outputs may be from about 0.1 seconds to about 10 seconds. Certain components of the optical spectrum can be applied for different times, powers, or energies. In the case where multiple light sources are used, one or more light sources can be powered off after its energy density is provided or achieved.

Energy density, fluence, or other dosage parameter, such as, for example, power, energy, illumination, or irradiance, may be measured at any of a variety of positions with respect to the tip 118 of the therapy device 100. For example, in one embodiment, fluence is measured substantially at the tip 118 of the therapy device 100. In this case, the dosage at the illumination surface is the fluence multiplied by the fluence area (for total power) and then divided by the illuminated surface area (e.g., in the nasal cavity, the surface area can range between 10 and 70 cm² as taught by Koreck). Therefore to achieve the desired dosage density, the fluence at the tip is approximately the dosage multiplied by illuminated surface area and then divided by the tip area. In another embodiment, the fluence is measured at a distance of about 0.5 cm, about 1 cm, or about 2 cm from the surface of the tip 118 of the therapy device 100.

The particular nasal cavity being treated may determine the energy density or dosage requirements. If the lining of the cavity is particularly far away from the therapy device 100, a higher energy, fluence, or intensity may be chosen. In the case where the nasal cavity is being treated and rhinitis is the disease, the dosage from the tip 118 may be chosen appropriately. For example, it has been shown by in-vitro work that T-cells undergo apoptosis at energy densities of about 50-100 mJ/cm² of combined UVA, UVB, and white light. The energy densities exiting from the tip of the therapy device used to achieve such energy densities as measured at the treatment site, may be 5-10 times this amount because of the distance of the optical therapy device 100 from the treatment site during treatment.

The energy densities may be further increased from that achieved in-vitro because of intervening biologic materials that may absorb light. For example, the mucus, which is present on top of the nasal mucosa in all patients, may absorb light in the desired region of the spectrum. In this case, the fluence or output of the therapy device 100 at the tip 118 can be corrected for the extra absorption. Furthermore, the mucosa may absorb more or less light at different time points during an allergy season (for example) and therefore the fluence of the therapy device may be controlled at these times. In many embodiments, this control is provided by the therapy devices. Photoreflectance data from the mucosa can be used by the patient, the medical practitioner, or automatic feedback (e.g., from the tip 118) to a controller and/or data processor. Such data can be used to estimate the thickness of the mucus layer and adjust the output of the therapy device 100 accordingly. In addition, the practitioner can evaluate the mucosa visually with a rhinoscope or speculum and adjust the optical parameters accordingly; in another embodiment, insertion member 106 delivers an image from the region surrounding the distal tip 118. Such an image can be captured and transmitted electronically, for example, via charge coupled devices (CCD) or CMOS devices. In other embodiments, an endoscope is used to provide images.

The dosage may be measured at a planar or curved surface with respect to the tip 118 of the therapy device 100. For example, in one embodiment, the dosage is measured at a plane that is tangential to the surface of the tip 118 of the therapy device 100. In another embodiment, the dosage is measured at a plane that is a distance of about 0.5 cm, 1 cm, 2 cm, 3 cm or 5 cm from the surface of the tip 118 of the therapy device 100. In another embodiment, the dosage is measured at a partially spherical plane that is at least partially tangential to, or at a distance of about 0.5 cm, 1 cm, 2 cm, 3 cm or 5 cm from the surface of the tip 118 of the therapy device 100. The selection of planar or curved surface for dosage measurement, and the distance between the measurement plane and the therapy device 100 tip 118 may be selected based upon the particular geometry of tip 118 utilized.

In one embodiment, the output portion 130 of the light source 126 is positioned so that it resides within at least a portion of the insertion member 106. When the output portion 130 of the light source 126 is so positioned, light emitted from the light source 126 is transmitted directly into the insertion member 106. In this embodiment, the insertion member is a reflecting tube. In such a case, optical losses may be minimized, or reduced. In addition, by positioning the output portion 130 of the light source 126 inside of the insertion member 106, additional optical focusing elements, such as lenses or mirrors, may not be required; moreover, the geometry of the insertion member can be optimized, such that light transmission is optimized by for example, creating surfaces within the insertion member designed to reflect light through and along the insertion member to transport the light to the distal end of the insertion member. In addition, the insertion member can be created to optimize total internal reflection of the light from the light source.

In some embodiments, the insertion member 120 includes one or more optical fibers that capture and guide the light from the light sources 126. When the light sources 126 are small semiconductor structures, the fibers can encapsulate the semiconductor structure and faithfully transmit substantially all of the light from the light source 126. More than one fiber can be used to direct the light from multiple light sources 126. For example, each fiber can transmit light from one light source 126. In other embodiments, the insertion member 106 is or includes a light guide such as a liquid light guide (e.g., such as those available from EXFO in Ontario, Calif.).

The insertion member 106 may taper from a large diameter at its proximal end 108 to a smaller diameter at its distal end 116, in which case the insertion member 106 has a larger diameter at its proximal end 108 than at its distal end 116. In another embodiment, the insertion member 106 may taper from a larger diameter at its distal end 116 to a smaller diameter at its proximal end 108. In such case, the insertion member 106 has a larger diameter at its distal end 116 than at its proximal end 108. In other embodiments, the insertion member 106 is substantially cylindrical. In such case, the diameter of the insertion member 106 may be substantially constant along its entire length.

In one embodiment, the insertion member 106 is flexible so that its shape and orientation with respect to the housing or hand piece 102 may be adjusted. A flexible material, such as rubber, plastic, or metal may be used to construct the insertion member 106, and to provide flexibility thereto. In one embodiment, a goose-neck tube, or spiral wound coil is used to provide a flexible insertion member 106.

An outer sheath 142 with tip 140, as further illustrated in FIG. 2B, may be provided to at least partially cover the insertion member 106. In one embodiment, the sheath 142 is disposable and in another embodiment, the sheath is not disposable. As will be appreciated by those skilled in the art, disposable components provide a convenient means to prevent cross-contamination between patients. Alternatively, the sheath 142 is reusable and will be sterilized, disinfected or cleaned between uses.

The sheath 142 is sterilizable and in other embodiments, the sheath 142 is not sterilizable. Sterilizing methods include, without limitation, ethylene oxide (ETO), autoclaving, radiation, and cold soak sterilization. Alternatively, the sheath 142 can be disinfected or cleaned with a disinfection solution (e.g. Cidex™), soap and water, or isopropyl alcohol.

The sheath 142 or the sleeve can be molded, thermoformed, machined or extruded. As will be appreciated, the sheath 142 can be made from any of a variety of biocompatible materials well-known in the art such as, but not limited to, PTFE, ePTFE, FEP, PVDF, urethane, polyethylene, polypropylene or silicone rubber. The tip 140 can be an optically transparent material or can have an open configuration where the light diverges as it leaves the sheath 142. The tip 140 can be produced from different materials. In this embodiment, the inner material will transmit light without absorbing the light. These configurations generally allow optical energy, or light, generated by the light source 126 to travel through the insertion member 106 and exit both the tip 118 of the insertion member 106 and the tip 140 of the sheath 142. In such embodiments, light energy is emitted from the therapy device 100 and absorbed by the tissue within the nasal cavity of the patient's nose.

The outer sheath 142 can also have beneficial optical properties. For example, the outer sheath can diffuse or otherwise pattern the light entering it from the insertion member 106. The outer sheath can be made of more than one material. For example, in some embodiments, the portion of the sheath where the light exits, or tip, 140 can be produced from an optically transparent material such as UV transmissive silicone, UV transmissive acrylic, fused silica, or quartz, and the portion which surrounds insertion member 106 can be produced from a biocompatible material which does not necessarily transmit ultraviolet light.

In one embodiment, the insertion member 106 is sized so it may be inserted into a cavity of a patient or user. For example, when the insertion member 106 is inserted into the nasal cavity until its tip 118 reaches the turbinates, the sinuses, or the ostia to the sinuses. The insertion member 106 may be made of flexible materials so that it can bend, or be steered around corners, or conform to the shape of the cavity, as required.

The insertion member 106 may be made from any one or a combination of materials as described above. For example, the insertion member 106 may be made from polymers. In such case, since many polymers absorb light in the ultraviolet portion of the spectrum, the inside wall 120 of the insertion member 106 may be coated with a reflective coating or layer 122, as described above. The outside of the insertion member 106 can also be coated with a polymer with the inner material being one of the materials noted above. Regardless of the inner material from which the insertion member is produced from, the outer portion of the insertion member (or the outer sheath 142 in the case when an outer optical sheath is used), particularly the distal end, can be coated or covered with a material which provides enhanced tactile feedback to the operator or enables the operator to move structures (for example, wedging open the nasal valve) in a comfortable way for the patient. The coating or covering can be a viscoeleastic material (e.g. a hydrogel or biologic such as collagen), a hydrophilic or hydrophobic coating or covering, or polymer, rubber or other elastomeric material. The coating or covering can be in the form of a pullover sheath 142 or can be more integral (e.g. formed via deposition processes) to the insertion member. In other embodiments, the insertion member has a joint at its distal end 116 so that the distal end can pivot with respect to the insertion member. Such a pivot would also facilitate placement of the device into the nasal cavity as well as its movement within the nasal cavity.

The controller 136 of the therapy device 100 is adapted to control the quantity (e.g., total energy) and intensity (e.g., power) of light emitted by the light source 126 and thereby exiting the tip 118 of the therapy device 100. For example, in one embodiment, the controller 136 determines and/or controls the power from the power supply 110. The controller 136 may be programmed and may include a timer so that only a pre-specified amount of light can be provided by the therapy device 100 at any given time, and such that a user cannot receive more than a predetermined dose in a specified short time period (e.g., over a period of one day) or a number of doses in a specified time period (e.g., over a period of months, for example). The controller 136 can also be configured to control the illumination pattern. For example, by turning one or more light sources powered on and powered off, the illumination pattern can be controlled. The controller 136 can further control the illumination pattern by moving (actively or passively) or otherwise altering the aperture or pattern of the tip 118. The controller 136 can also apply current to the light sources at a desired frequency or duty cycle.

In another embodiment, the controller 136 is adapted to deliver a large current or a current or voltage pulse to the light source 126 to “burn out” or destroy the light source 126 after a selected period of time. For example, after a predetermined “useful lifetime” of the therapy device 100 expires, a “burn out” current is provided and the therapy device 100 essentially ceases to function. At this time, the therapy device 100 is discarded. The controller 136 can also respond to or receive a control signal from one or more photodetectors placed in or on the insertion member 106 or the controller can respond to receive a control signal from one or more photodetector devices in an external calibration unit.

The power supply 110 of the therapy device 100 is adapted to receive power from an alternating current (AC) source, a direct current (DC) source, or both depending on the number and types of light sources. For example, in one embodiment, power supply 110 includes a battery, battery pack, rechargeable DC source, capacitor, or any other energy storage or generation (for example, a fuel cell or photovoltaic cell) device known to those of skill in the art. In some embodiments, an LED may utilize a DC power source whereas a mercury vapor lamp may utilize an AC power source. Thus, where a device is configured to use more than one light source, it may be necessary to provide more than one power source.

The light source 126 in any of the embodiments described herein can include or be selected from light emitting diode (LED) such as the UV LED manufactured by S-ET Corporation (Columbia, South Carolina), which can be produced to emit narrowband light at any wavelength from about 250 nm to 365 nm. More specific wavelength, such as a wavelength of 275 nm, can be used. In such cases, the UV LED may have a sapphire substrate with conductive layers of aluminum gallium nitrite. For example, in one embodiment, the UV LED has about 50% aluminum. By varying the concentration of aluminum, the wavelength peak can be adjusted. In some embodiments, the several LEDs are packaged together such that light output with multiple peaks in the ultraviolet range can be achieved. In some embodiments, the aluminum concentration is varied along a dimension of the chip such that a more continuous spectrum is achieved when current is passed through the chip. In addition, the UV LED packaging may include flip-chip geometry. In such case, the LED die is flipped upside down and bonded onto a thermally conducting sub-mount. The finished LED is a bottom-emitting device that may use a transparent buffer layer and substrate.

In such embodiments, the light is two-times brighter when the LEDs are in a flip-chip geometry. This is due to the fact that light emitted from the LED is not physically blocked by an opaque metal contact on the top of the LED. In addition, flip-chip sub-mount pulls heat away from the device when made from materials having high thermal conductivity. This improves efficiency levels with less energy being converted to heat and more energy being converted to light. The resulting device will have a lower weight, will be smaller, and will be resistant to vibrations and shock.

Power delivery to the LEDs can be modified to optimize the optical power of the LEDs. In such cases, the LEDs can be configured to switch on and off in order to prevent heat build up which would otherwise decreases the efficiency of the LEDs. For example, a temperature rise may decrease the potential optical power. Such switching can increase the power output several-fold. In other embodiments, the semiconductor structure takes the form of a laser diode module wherein the semiconductor package contains reflecting optics to turn the non-coherent light into coherent light.

Suitable sources of lights also include, for example, Nichia Corporation (Detroit, Mich.) supplies ultraviolet light emitting diodes which emit relatively monochromatic, non-coherent light in the range 365 nm to 400 nm. LED Light Corporation (Carson City, Nev.) sells high powered white light LEDs with output from 390 nm to 600 nm. Cree Inc. (Durham, N.C.) also produces and sells LED chips in the long wave ultraviolet as well as the blue, amber and red portions of the electromagnetic spectra.

As will be appreciated by those skilled in the art, a variety of suitable lighting sources can be employed in the embodiments disclosed herein, without departing from the scope of the invention.

Although the power supply 110 of the therapy device 100 is illustrated in FIG. 2A as tethered to the proximal end 112 of the hand piece 102, it should be well understood by those of skill in the art that the power supply 110 may be incorporated into or included on or within the body of the device, including the hand piece 102. In such cases, the power supply 110 may include a battery, a battery pack, a capacitor, or any other power source. The power coupling 114 in such embodiments may include contacts or wires providing electrical communication between the power supply 110 and the control circuit 134.

The optical emitter 128 of the light source 126 is generally in electrical communication with leads 132. The optical emitter 128 can be adapted to extend in a direction that transverses an axis of the light source 126. As will be appreciated, the optical emitter 128 schematically represents only one embodiment of the light emitting portion of the hand piece 102 and light source 126. Optical emitter 128 (e.g., the light emitting portion of the light source 126) can be made from any of a variety of materials known to those of skill in the art; in cases where the optical emitter 128 represents a wire-filament type light source, the optical emitter 128 can include tungsten.

Although the optical emitter 128 of the light source 126 is shown at the distal end 124 of the hand piece 102, in other embodiments, the optical emitter 128 is positioned closer to the proximal end 112 of the hand piece 102. By moving the optical emitter 128 proximal with respect to the tip 118 of the insertion member 106, heat generated by the light source 126 may be at least partially separated from the insertion member 106, thereby lessening thermal communication with the patient's tissues.

Heat generated by the light source 126 may be removed from the therapy device 100 by any of a variety of methods and devices known to those of skill in the art. For example, in one embodiment, heat is directed away from the hand piece 102 by convection or conduction. In other embodiments, active cooling devices, such as thermoelectric coolers or fans may be employed. Alternatively, or in addition, passive cooling structures, such as heat fins, heat conductors and/or cooling tubes may be used to remove heat from the therapy device 100.

In one embodiment, the light source 126 includes a solid state light emitter (e.g., an LED or laser diode module) and the light source 126 is positioned at or near the distal end 116 of the insertion member 106 instead of within the hand piece 102.

In another embodiment, the light source 126 includes a solid state emitter and a mercury vapor lamp (or other analog-type light source that emits ultraviolet light as described above). Such combinations may be useful to provide light of multiple wavelengths or intensities that correspond to select spectral peaks. Multiple solid state emitters may be employed to achieve the same or similar results. Additionally, a visible light solid state emitter is combined with a mercury vapor or halogen lamp to enhance wavelengths in the visible light region can be used. Alternatively, an array of solid state emitters may be arranged on an integrated circuit layout to produce spectral output that can be continuous or semi-continuous depending upon the wavelength, number and bandwidth of each emitter.

As discussed above in more detail, the insertion member 106 may include a soft coating on its outside surface 138. A soft coating, such as a polymer, rubber, or fluid-filled jacket, provides a comfortable interface between the outside surface 138 of the insertion member 106 and the patient's nose. In addition, the insertion member 106 may include one or more filters along its length. A filter can be positioned within the insertion member 106 near its proximal end 112 or near its distal end 116. The filter may function as a lens if cut into an appropriate shape and placed at the distal end 116 of the insertion member 106. One such optical filter well known to those of skill in the art is manufactured by Schott and includes glass optimized for absorption at certain wavelengths.

The light source 126 can have an efficiency ranging from about 1% to 40%, more typically from about 10% to 15%. The light source or combinations of light sources 126 can be configured to generate about 5 mW to about 100 mW of optical power. The light source can be configured to dissipate between about 5 W to about 200 W of power in order to generate about 5 mW to about 100 mW of optical power. Excess heat is typically dissipated so that the therapy device 100 does not overheat, and/or so that the patient does not experience discomfort during its use.

Heat transfer control may become increasingly important when the therapy device 100 includes a light source 126 that is located near the distal end 116 of the insertion member 106 (e.g., heat may be closer to the patient's tissue). For example, where the light source 126 is a mercury vapor light source, heat is generated near the output portion 130 where the mercury plasma is generated. Since, in this embodiment, most of the light generated is non-blackbody radiation, very little heat is generated as photons propagate towards the distal end 116 of the insertion member 106 and enter the tissue of the patient. Therefore, in such embodiments, heat transfer mechanisms are generally confined to the output portion 130 of the light source 126, close to where the light is generated.

In one embodiment, a fan is provided to transfer heat or to remove heat from the therapy device 100. The fan may be configured to surround at least a portion of the output portion 130 of the light source 126 or the entire light source 126 itself. Thus, the fan may surround the light source 126, or a portion thereof, in an annular fashion, and can direct heat away from the light source 126 and away from the patient via convection.

Alternatively, a heat tube is placed around the light source 126 and the heat tube directs heat away from the patient towards the proximal end 112 of the hand piece 102. At the proximal end 112 of the therapy device 100, heat may be released into the environment. The heat tube can be configured to terminate in a structure optimized for heat transfer into the surrounding environment, for example, cooling fins. Alternatively, or in combination, in another embodiment, a fan is provided at the proximal end 112 of the therapy device 100 and at the proximal end of the heat tube. The fan provides active convection to carry heat away from the therapy device 100.

A controller 136 can also be provided that is adapted to control the power output from the power supply 110 so that the light source 126 is activated for a predetermined time period. The controller 136 may include a switching mechanism that is controlled external to the device. Such external control may be implemented by any of a variety of mechanisms, such as, for example, a radio frequency communicator. The controller 136 helps avoid misuse or overuse of the therapy device 100. The controller 136 may also allow optimization to be carried out by the physician prescribing the device. In another embodiment, the controller 136 provides for preset dose quantity and frequency. These parameters can be set either automatically, semi-automatically or with user interaction. Thus, the parameters can be controlled by the patient's physician, a programmable controller, a nurse, caregiver, patient, or other individual, or may be set according to prescription set forth by clinician.

The therapy device 100 can be adapted to include software (not shown) to control the dosage of optical energy to a patient. Thus, the energy, power, intensity, and/or fluence of the optical output may be adjusted. Thereafter, adjustments and settings may be saved within or loaded onto the therapy device 100 to correspond to the requirements of a particular patient, or clinical result.

The treatment dose can be configured to include timing controls. Timing controls may include the amount of time the light source 126 of the therapy device 100 may be activated for a treatment. Timing controls include pulsing parameters, such as pulse width, timing of optical pulses, or relative sequence of pulses of light emitted from one or multiple light sources 126. Thus, the light source 126 can be adapted to provide continuous (non-pulsed) optical output, and the timing controls include the duration of treatment, the time between treatments, and the number of treatments allowed in a specified time period, for example, one day.

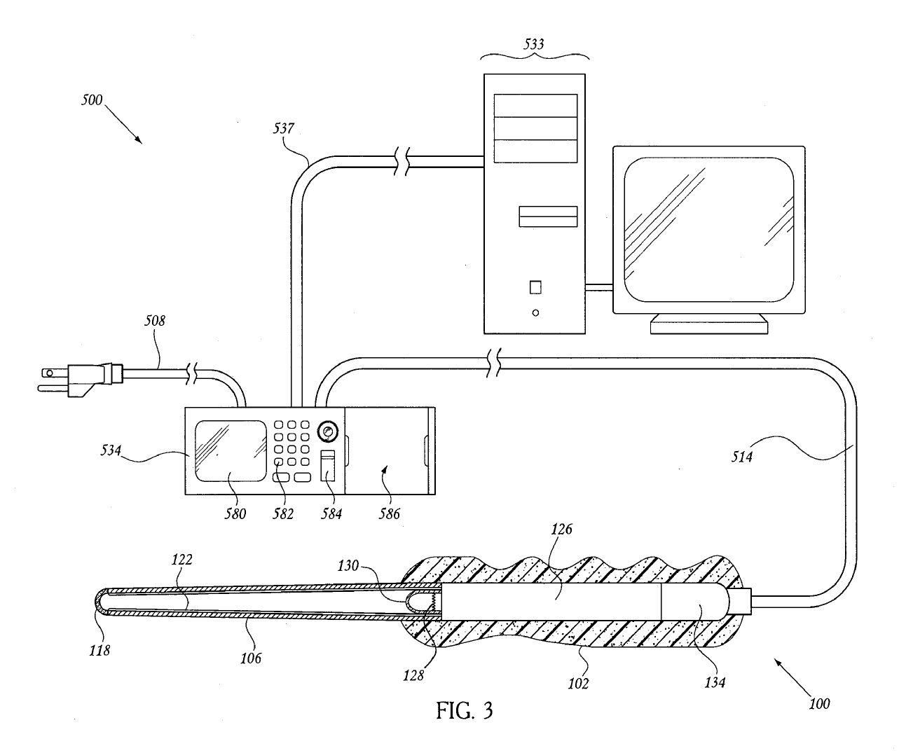

A therapy system 500 is illustrated in FIG. 3. The system, as with other devices and systems described, can be adapted for use with any of the embodiments described below. The therapy system 500 includes a therapy device 100, a control unit 534, and at least one computer 533. Control unit 534 communicates with therapy device 500 via power coupling 514, such as power coupling 514 described above. The control unit 534 is also coupled to at least one computer 535 via computer coupling 537. Computer 535 may include a personal computer, such as a PC, an Apple computer, or may include any of a variety of computing devices, such as a personal digital assistant (PDA), a cellular telephone, a BLACKBERRY™, or other computing device. Computer coupling 537 may be any of a variety of structures, devices, or methods known to those of skill in the art that enable communication between computers or computing devices. For example computer coupling 537 is a cable, such as a USB or Ethernet cable. The computer coupling 537 can also be a wireless link. Computer coupling 537 may include any wired or wireless computing connection, including a Bluetooth™, infrared (e.g., IR), radiofrequency (e.g., RF), or IEEE 802.11(a)-, (b)-, or (g)-standard connection. Control unit 534 and computer 533 may form a network within which multiple computers 535(1-n) or computing devices, or control units 534(1-n) may be included.

In one embodiment, control unit 534 is connected to a power supply via a power cord 508. Control unit 534 also generally includes a display 580, a keypad 582, controls 584, and a cradle 586. Display 580 may include a screen or other output device, such as indicators, lights, LEDs, or a printer. The display 580 can be a touch screen that includes touch controls to control the parameters of the therapy device 500. Controls 584 include any of a variety of input devices, including knobs, levers, switches, dials, buttons, etc. Cradle 586 can be adapted to receive the hand piece 502 of the therapy device 500 when not in use. Such a cradle 586 may furthermore be configured to provide electrical power (e.g., a rechargeable battery) to the hand piece of the therapy device 500 and/or control signals. Power coupling 514 may not be provided, or may be provided via the cradle 586 through electrical contacts. The cradle 586 can be adapted to include a detector, such as a photodiode, which can provide an indication of the output or strength of the light source 526 and can provide for calibration of the therapy device 500 over time.

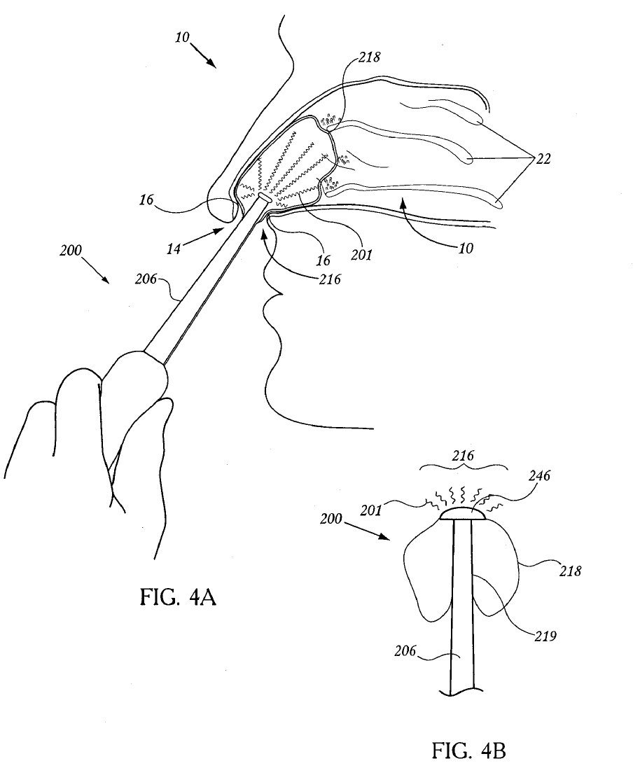

FIG. 4A depicts another embodiment of therapy device 200. The optical therapy device is adapted to provide therapeutic light to a target region of tissue within a nasal cavity. An expandable component 218 holds the therapy device in place in a space between a nasal septum and a lateral wall of the nasal cavity while the phototherapy is being delivered posterior to the limen nasi. This embodiment enables a light source to illuminate a target region of tissue within the nasal cavity while preventing illumination of a region of tissue anterior to the nasal cavity. The expandable component 218 enables targeting of the light by expanding within the cavity. When expended, the expandable component registers the position of the insertion member relative to the nasal anatomy to facilitate positioning of the distal end. Thus, the expandable component 218 can be positioned within the nasal cavity 10 to allow the therapeutic light to be applied to one or more target regions of the nasal cavity. Because the expandable component 218 grips the walls of the nasal cavity, the therapy device can pivot and deliver light to various parts of the nasal cavity 10 without delivery light to other regions of the nasal cavity. The expandable component can be, but is not limited to, a balloon, a wedge, and a custom-molded elastomer. The expandable component 218 can expand the limen nasi 16 or other portions of the nasal cavity by either an expanding or wedging action. Additionally, this allows the therapy device 200 to pivot and change direction in the nasal cavity 10 without mechanically irritating the sides of the nasal cavity. In some embodiments, the device is adapted to enable positioning within a nasal cavity without visualization of the nasal cavity by the operator, e.g. during insertion.

The insertion member is adapted to deliver therapeutic light from a light source to a region of the nasal cavity, such as the respiratory epithelium, and to prevent light delivery to another region, such as the squamous epithelium of the nasal vestibule. Preventing light delivery to, for example, the nasal vestibule, nasal septum and/or nasal valve, prevents erythema to the squamous epithelium of the patient undergoing treatment and/or to tissue which does not respond to the therapy.

In some embodiments, the device can be adapted to provide a sensor, as described further below, that provides nasal structure feedback to an operator. Feedback can also be visual feedback from an imaging element, such as a semiconductor imagine element.

The expandable component 218 can be positioned at the distal end 216 of the elongate body 206 such that it extends proximally and distally with respect to the distal end 216. Alternatively, the expandable component 218 can be positioned only distally with respect to the distal end 216. In some embodiments, such as that in FIG. 4A, the light 201 is emitted within the expandable component 218. In such embodiments, the expandable component 218 generally includes at least a portion that is at least partially transparent to the therapeutic light wavelengths of the therapy device 200.

In the case where the expandable component 218 is a balloon, the therapeutic light is delivered to the tissue through the wall of the balloon. In one embodiment, the balloon is used to compress the mucous of the nasal cavity and deliver a more relatively uniform distribution of light.

The therapy device 200 is inserted into the nasal cavity 10 such that the expandable component 218 resides at least within the vestibular portion 14. When the expandable component 218 is expanded, or if not expandable, when otherwise positioned and inserted into the nasal cavity 10, the expandable component 218 can wedge open the vestibular portion 14 of the nasal cavity 10, which does not have respiratory mucosa. In some embodiments, the expandable component 218 does not expand and is positioned within the nasal cavity 10 such that the therapeutic light reaches one portion of the nasal cavity but shields another portion from the therapeutic light. For example, in one embodiment, light reaches the turbinates 22 but not the vestibular surface where there is no respiratory epithelium and which will have a very different response to the therapeutic light. The expandable component 218 can be filled or expanded prior to placement in the nose or after insertion into the nasal cavity 10. In the case where it does not expand, the expandable component 218 can wedge open a portion of the nasal cavity as it is being positioned.

Further to the device depicted in FIG. 4A where the therapeutic light emitted from distal end 216 and travels through the expandable component 218, the expandable component 218 can include an opaque portion and a transparent portion. The opaque portion blocks light 201 emitted from the distal end 216 of the therapy device 200 and prevents light from being absorbed by a predetermined portion of the nasal cavity 10. For example, in the illustrated embodiment, a proximal portion of the expandable component 218 is the opaque portion which blocks light from being absorbed by the vestibular portion 14 of the nasal cavity 10. A distal portion of the expandable component transmits the therapeutic light 201 through the wall of the expandable component and to the tissue of the nasal cavity 10. Additionally, the distal end can be configured to condition the light emitted from the insertion member, for example by the use of scattering particles, a lens, a filter, a mirror or a diffuser. In some embodiments, the device can be adapted to provide imaging information from the interior of the nasal cavity to an operator. Depth gauges and/or rulers can also be included in the design to further facilitate the ability to determine how far into the nasal anatomy the distal end of the device is positioned.

In another embodiment, as shown in FIG. 4B, the expandable component 218 of therapy device 200 can be positioned at the distal end 216 such that it extends only proximally with respect to the distal end 216. In this embodiment the expandable component surrounds the distal end of the optical therapy tip and can be opaque because the light radiates from the device into the cavity and not through the expandable component. The expandable component 218 can be adapted to have an annular configuration and a lumen 219 through which the elongate body 206 of the therapy device 200 extends. Light 201 is emitted through an emitter from the distal end 216 of the elongate body 206. The distal end 216 of the elongate body 206 can also include an atraumatic tip 246. The expandable component 218 of the therapy device 200 can hold the therapy device 200 in place while phototherapy is delivered to the nasal cavity 10 from the therapy device 200. Since the expandable component 218 is usually produced from a flexible or compliant material, such as a balloon, the therapy device 200 is able to pivot and change its orientation in the nasal cavity 10. In addition, the expandable component 218 allows the therapy device 200 distal end 216 to be manipulated within the nasal cavity 10 without mechanically irritating the sensitive soft tissue located at the region of the vestibule. In use, the user inserts an insertion member (such as the distal end of the therapy device) into the nasal cavity to selectively deliver a light into the nasal cavity. Light is delivered by use of, for example, an emitter. The distal end of the device is positioned such that the device illuminates a target region of tissue at or posterior to the limen nasi of the nasal cavity (i.e., the distal end of the device is advanced into the nasal cavity such that the distal end, such as the tip, is positioned within the nasal cavity at or beyond the portion of the nasal anatomy known as the limen nasi). The design of the device is such that the distal tip of the device can be positioned within the nasal cavity at or distal to the limen nasi. Once the distal tip of the device is positioned within the nasal cavity at or distal to the limen nasi, phototherapy is then delivered to a target region of the nasal cavity. The configuration of the device is such that the expandable component 218 operates to wedge open the limen.

Where the distal tip incorporates a balloon, the balloon can be fillable or optionally not fillable. If fillable, then a fluid transmission line is used to fill the balloon. If filled prior to placement in the nose, the therapy device is wedged into the nasal vestibule up to the region of the limen nasi. If filling is required, then the balloon is placed at the region of the limen nasi and then expanded with a fluid. Typical expansion fluids are water, air, saline, a hydrogel, or any material with sufficient viscosity so that the balloon conforms to the anterior portion of the nasal cavity. Although a balloon is shown in this embodiment, any type of soft semi-compliant material (e.g. foam, rubber, elastomer, hydrogel, etc.) can be used as long as there will be a wedge action to push apart the lateral wall of the vestibule from the nasal septum. A typical silicone balloon filled with water satisfies this requirement. As with the previous design, the design is adapted to enable the device to deliver therapeutic light within the nasal cavity with or without visualization of the distal tip posterior to the limen nasi of the nasal cavity. As described above and below, the compliant material prevents trauma to the walls of the nasal cavity.

Once the device has been advanced into the nasal cavity, one or more areas of the nasal cavity can be treated by, for example, advancing the device into or withdrawing the device from the nasal cavity. By moving the device, or a portion thereof, into the nasal cavity or withdrawing the device, or a portion thereof, the depth of a distal tip within the cavity can be adjusted.

The atraumatic tip 246 can be made from a material which is optically transparent to the light emitted from the therapy device 200. In addition, the atraumatic tip 246 can be designed to scatter, focus, or otherwise condition the light exiting the therapy device 200 distal end, as desired. The atraumatic tip can have a smooth and/or soft surface so that it does not irritate the inside wall of the nasal cavity 10 when inserted and manipulated therein.

The expandable component 218 can be made from a completely opaque material so that it blocks light emitted from the therapy device from being absorbed by the epithelium of the nasal cavity 10 at the region where the expandable component 218 contacts the nasal tissue. As in the embodiment of FIG. 4A, the expandable component 218 holds, or otherwise positions, the therapy device 200 in place on the region in the nasal cavity 10 while phototherapy is delivered. This allows the therapy device 200 to pivot and change direction and orientation within the nasal cavity 10 without mechanically irritating the nasal cavity 10. Although in one embodiment, the expandable component is made from an opaque material to prevent light from reaching the area touched by the expandable component, the expandable component can be made from an optically transparent material. In this case, the therapy device, by virtue of the expandable component positioning the therapeutic light such that the light is always directed forward into the nasal cavity, illuminate only the regions intended to receive therapeutic phototherapy.

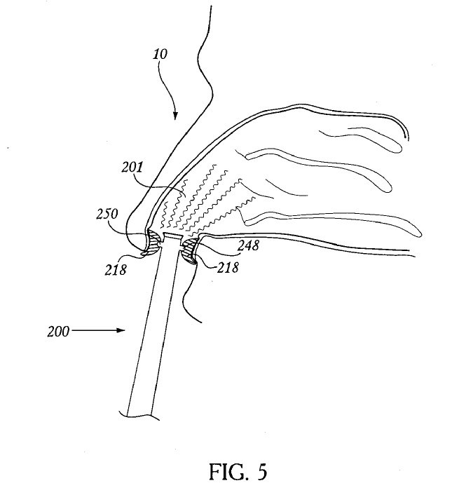

FIG. 5 illustrates an optical therapy device having a light source communicating with a distal end of an insertion member to deliver light to a target region of tissue within a nasal cavity posterior to a limen nasi and to prevent illumination of a region of tissue anterior to the limen nasi according to another embodiment. The optical therapy device of the embodiment can also be configured so that the insertion member is adapted to deliver light from a light source to a first region of the nasal cavity, such as the respiratory epithelium, while preventing light delivery to a second region, such as the squamous epithelium, among others. The optical therapy device 200 has an expandable component 218 includes a mating portion 248 that is adapted to be removably coupled to the insertion member at a distal end of a therapy device 200. The expandable component 218 can be adapted to expand the space between the nasal septum and the lateral wall of the nasal cavity and/or to partially blocking light from the light emitters. Additionally, the expandable component can be adapted to engage other embodiments of optical therapy devices disclosed herein. The mating portion 248 can be a clip, o-ring, band, snap, thread, groove, recess, or any other suitable mating portion 248. The therapy device 200 has a corresponding mating portion 250 to mate with the mating portion 248 of the expandable component 218. The mating portions 248, 250 allow the expandable component 218 and therapy device 200 to be removably coupled to each other. The mating portion can be inserted into the nasal cavity prior to the device and then the device can be coupled to the mating portion. In some embodiments, the mating portion 250 is a friction mate. In one embodiment, the mate is a sticky material or has a sticky material attached to assist in the mate. The therapy device 200 is placed in the expandable component 218 and held in place by a frictional force between the therapy device 200 and the mating portion 248.

As with other embodiments, the device 200 can also include a light conditioner configured to condition light. Suitable light conditioners include, for example, light scattering particles, lenses, filters, mirrors, and a diffuser. In some embodiments, the insertion member can be further configured to provide nasal anatomy information to the operator, such as by providing imaging information. Additionally, the device can be adapted to provide a depth gauge, or ruler, to set the depth of light delivery, as discussed in further detail with respect to other embodiments. The device can also be adapted to provide nasal structure feedback to an operator, such as feedback from an imaging element, such as a semiconductor imaging element.

The expandable component 218 is inserted into the nasal cavity 10 and positioned so that it is comfortable for the patient and protects the region of the nasal vestibule. As above, the expandable component 218 does not have to actually expand tissue but can act to position the device without expanding. The expandable component 218 is typically made from a soft elastomeric material, such as rubber, polymer, ePTFE, a hydrogel, or any other soft comfortable material. The expandable component 218 can have elastic properties so that it can expand to conform to the inside shape of the anterior portion of the nasal cavity 10. The inside surface of the expandable component 218 conforms to the outside surface of the therapy device 200. When attached (e.g., friction coupled), the expandable component 218 can block or filter light emitted from the therapy device 200 from being absorbed by the tissue in the vestibular portion of the nasal cavity 10. In addition, the expandable component 218 can act as a pivot about which the distal portion of the therapy device 200 can be manipulated, rotated, translated, and/or moved. As in the expandable components described above, the expandable component protects the tissue of the nasal cavity 10 from the therapeutic light 201 and mechanical irritation from the distal end of the therapy device 200. The expandable member 218 can be configured to engage the insertion member in such a way that the depth of the insertion member is controllable or adjustable. Additionally, the expandable member 218 can be configured to be positionable relative to the nasal bridge of a patient. In some embodiments, the expandable member 218 can be further configured such that it determines or controls an angle at which the insertion member delivers optical therapy.

In another embodiment, the vestibular region of the nasal cavity is protected from the therapeutic light by placing a light absorbing substance on the surface of the vestibule of the nasal cavity. One example is a sunscreen but any substance which is opaque to white light and/or ultraviolet light can be used to protect the vestibular region of the nasal cavity from the therapeutic light. The light absorbing substance can be applied with a medical instrument, can be applied with the therapy device itself, or can be applied with a nasal spray, syringe applicator, or a finger. In one embodiment, the light absorbing substance is applied to the therapy device and therefore is applied as the device is used.

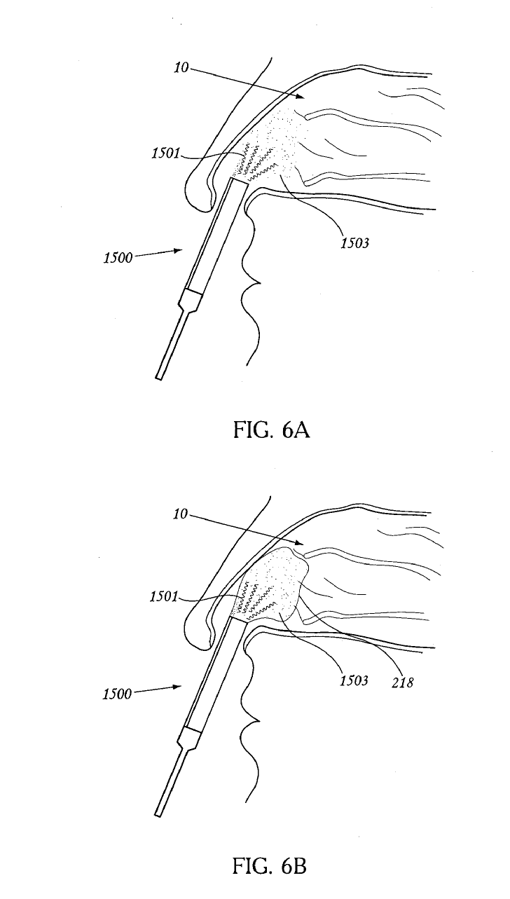

In the device 1500 depicted in FIG. 6A-B, the light 1501 is delivered from an optical therapy device. The device is configured to provide an insertion member that communicates with a distal end of an insertion member to deliver light to a target region of issue within a nasal cavity posterior to a limen nasi while preventing illumination of a region of tissue anterior to the limen nasi. As depicted, light is then scattered in the nasal cavity 10 with a multitude of particles 1503. The insertion member depicted can also be adapted to deliver therapeutic light from a light source to, for example, the respiratory epithelium, while preventing light delivery to, for example, the squamous epithelium, without the operator having visual access to the nasal cavity. Other embodiments of the devices of FIG. 6, can be adapted to provide a sensor to provide nasal structure feedback to an operator, such as by use of visual information from an imaging element or by way of a semiconductor imaging element. Particles 1503 could diffract or reflect light causing a broader or more even light distribution than is capable with a direct light beam. FIG. 6A illustrates an embodiment where particles are emitted from the distal end of the device into the nasal cavity. Light 1501 could be emitted concurrently with the particles 1503 or at separate times. Suitable particles 1503 may include, for example, saline mist. Other biocompatible particles can be used as can be appreciated by those skilled in the art.

Alternatively, as illustrated in FIG. 6B, light scattering particles could be contained in an expandable component 218 to act as a light conditioner Other light conditioners could be used without departing from the scope of the invention. As described above, the expandable component 218 could be fluid filled balloon or other soft, expandable component. Light scattering particles 1503 could be suspended or embedded in the expandable component 218. The particles 1503 may include, but are not limited to, such materials as biocompatible metals or plastics. Suitable metals might include stainless steel, titanium or aluminum. Suitable plastics might include PTFE which is a commonly used diffusing material in optical applications. In the case of a gas filled balloon, particles 1503 could be saline which would function to diffract and/or reflect the light 1501.

As will be appreciated by those skilled in the art, upon review of the disclosure, other features can be incorporated into the design of the embodiments of FIG. 6, as desired. For example, a depth gauge, or ruler, can be provided to assist in determining the depth of light delivery; the device can be adapted to be positionable relative to the nasal bridge; separable components can be provided that are positioned between the lateral wall of the nasal cavity and the nasal septum into which the device docks; the insertion member can be configured to transmit imaging information and/or nasal anatomy information to an operator. The insertion member of the devices can also be configured to expand a space between a nasal septum and a lateral wall of the nasal cavity.

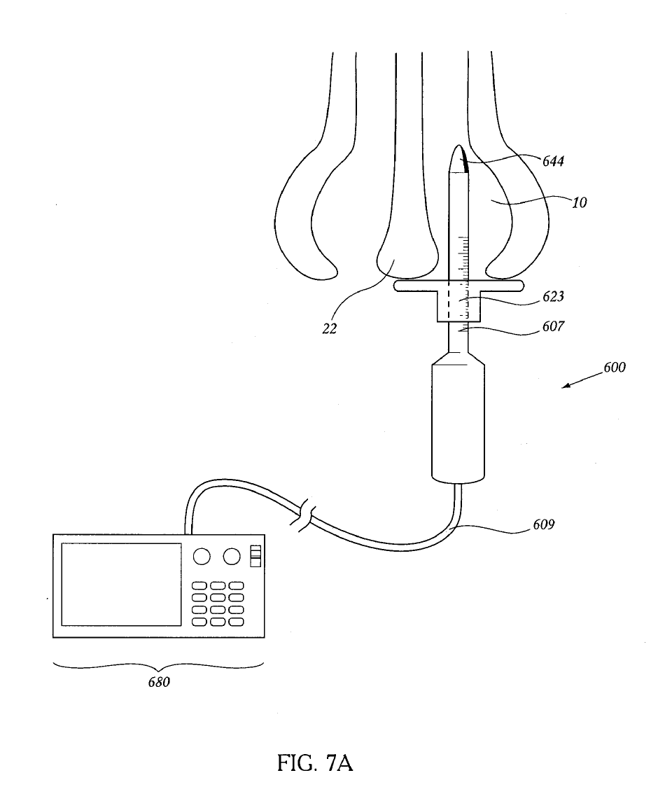

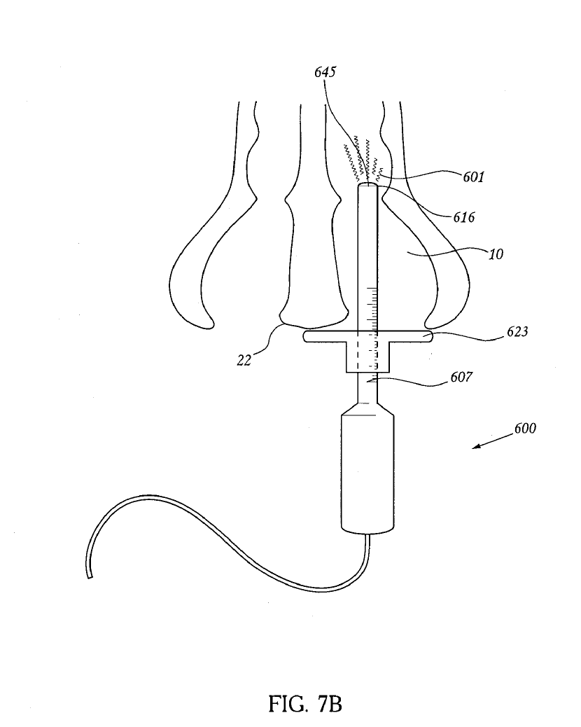

FIG. 7 illustrates another integrated measurement/positioning and therapy device 600 wherein the device is placed relative to the nasal cavity 10. In one embodiment, the device 600 is configured to provide an insertion member and a light source that is adapted to communicate with a distal end of the insertion member to illuminate target tissue within a nasal cavity posterior a limen nasi of the nasal cavity, such as respiratory epithelium, and to prevent illumination of non-target tissue, such as squamous epithelium. In another embodiment, the device 600 can be configured to provide one or more light sources and an insertion member that delivers therapeutic light to a target area of the nasal cavity while preventing light delivery to a non-target area without visualization of the nasal cavity by an operator. Still another embodiment, provides an insertion member having sensors adapted to provide nasal structure feedback to an operator and one or more light sources adapted to illuminate the nasal cavity.

More specifically, as depicted, in FIG. 7A, the device 600 is dimensioned to provide a gauge 607 for measuring the depth of the nasal cavity 10. The depth gauge can be configured to mark the depth of one or more structures, such as the nasal valve, the transition zone between the respiratory epithelium and squamous cell epithelium, and the vestibular limen. An example, of a suitable gauge would be a ruler with markings along its length that enables the user to measure anatomic relationships. By measuring the device, the user can avoid illumination of the anterior vestibule and the nasal valve and can focus the therapeutic light posterior to the limen nasi of the nasal cavity.

A sensor 644 can further be used to detect the narrowing of the nasal valve from the vestibule. In an embodiment employing a sensor, the sensor could be acoustic, infrared, temperature, humidity, electrical, pressure, or any other sensor suitable for enabling the device to detect the position of the valve. The depth determination could be made to assess depth relative to, for example, a physiological marker such as a nasal bridge 22. A stopper 623 could be used to position the device 600 against the nasal bridge 22. Other relative markers would be apparent to those skilled in the art and include, for example, other portions of the surface of the nose or face. The distance could further be determined with the use of visual markings or integrated electronics such as a linear potentiometer. As with previous embodiments, the device 600 can be adapted to interface with a display 680 by a cord 609. A power supply can also be provided either within the device (e.g., batteries located in the handle) or by an external power supply (e.g., a power cord). Other sensors 644 can be provided that are adapted to provide a depth determinative relative to a nasal bridge 22. The device is configured to account for physiological differences to ensure that the device is positioned within the cavity at a predetermined depth. As illustrated, a sensor 644 is inserted and provides feedback to an operator of the depth of the device and/or the position of the valve or limen nasi.

FIG. 7B illustrates another integrated measurement/positioning and therapy device wherein the device 600 adapted to use a gauge 607 to measure a depth of the nasal cavity. In FIG. 7B a device 600 is depicted in combination with a portion of the nasal cavity 10. The device 600 is adapted to provide a stopper 623. The stopper 623 is configured such that a portion of it abuts against the outer surface of the nose. An external anatomical marking, such as the alar, can be used to facilitate locating the nasal valve. Depth markings are provided along at least a portion of the length of the device to provide an indicator of how far into the nasal cavity 10 the device has traveled. Therapeutic light 601 is delivered from the distal end 616 of the device 600. The therapy can be light 601 that exits through a light exit 645. As discussed above, the light exit can be adapted into a variety of configurations that enable light conditioning or control. A predetermined depth could be selected prior to activation or treatment. Depth markers can be used to determine whether the appropriate or desired depth has been achieved. Additionally, the tip to stopper distance could be adjustable to accommodate anatomical variations.

Similar to other embodiments, it will be appreciated by those skilled in the art, upon review of the disclosure, other features can be incorporated into the design of the embodiments of FIG. 7, as desired. For example, the device can be adapted to be positionable relative to the nasal bridge; separable components can be provided that are positioned between the lateral wall of the nasal cavity and the nasal septum into which the device docks; the insertion member can be configured to transmit imaging information and/or nasal anatomy information to an operator. The insertion member of the devices can also be configured to expand a space between a nasal septum and a lateral wall of the nasal cavity. The devices can also be configured to dock within a separate device, similar to other embodiments depicted.

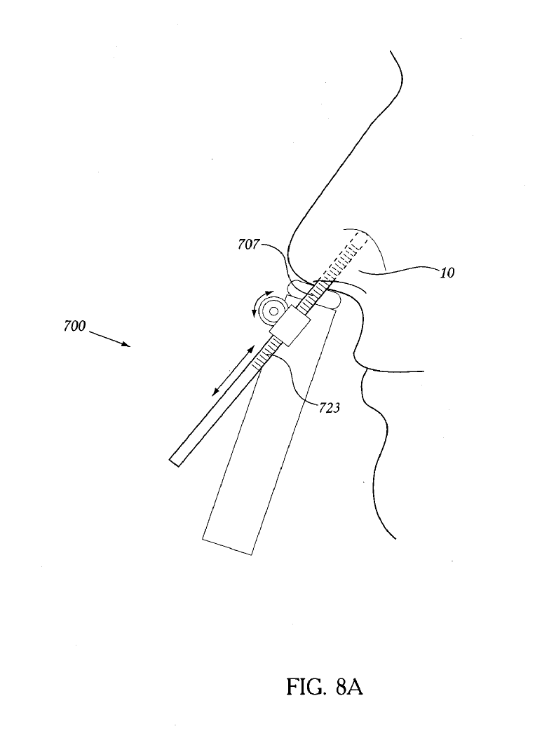

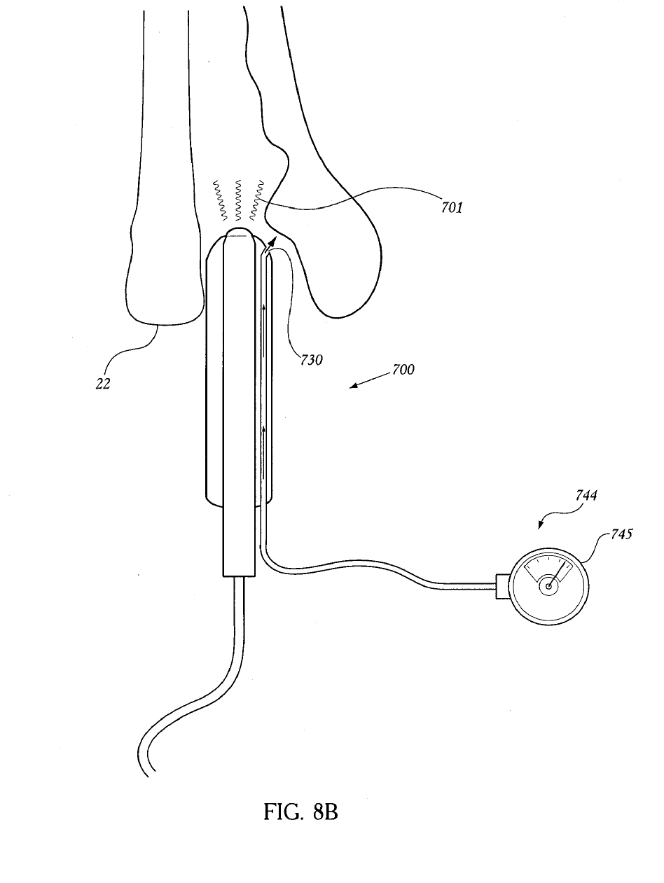

FIG. 8 illustrates yet another therapy device 700 wherein the device is placed relative to the nasal cavity 10. As with the previous embodiment, in one embodiment, the device 700 is configured to provide an insertion member and a light source that is adapted to communicate with a distal end of the insertion member to illuminate target tissue within a nasal cavity posterior a limen nasi of the nasal cavity, such as respiratory epithelium, and to prevent illumination of non-target tissue, such as squamous epithelium. In another embodiment, the device 700 can be configured to provide one or more light sources and an insertion member that delivers therapeutic light to a target area of the nasal cavity while preventing light delivery to a non-target area without visualization of the nasal cavity by an operator. Still another embodiment, provides an insertion member having sensors adapted to provide nasal structure feedback to an operator and one or more light sources adapted to illuminate the nasal cavity.

More specifically, as depicted in FIG. 8A, another embodiment of a device 700 has a custom depth gauge 707 adapted to control the depth at which therapy is delivered. The device 700 features a depth gauge 707 with markings 723 along the length to provide an indication of depth. The device is dimensioned such that it is placed against the nasal bridge and advanced into the nasal cavity. Additionally the gauge 707 can be adapted to use a rack and pinion mechanism, or slide mechanism, to control operation and to advance the distal end of the device into the nasal cavity.

In FIG. 8B a device 700 is illustrated that is adapted to provide a sensor 744 that detects air flow or pressure change. The distal tip of the device can be further adapted to provide an aperture or port 730 which enables air flow into or out of the tip. When the tip is inserted into the nasal cavity to a point where the port 730 aligns with the nasal valve, airflow and/or pressure through the port will change and the change in airflow or pressure will be measured by a gauge 745. The change in airflow or pressure acts as an indicator that the distal tip of the device is at a desirable depth for delivery of light 701.

Similar to other embodiments, it will be appreciated by those skilled in the art, upon review of the disclosure, other features can be incorporated into the design of the embodiments of FIG. 8, as desired. For example, a depth gauge, or ruler, can be provided to assist in determining the depth of light delivery; the device can be adapted to be positionable relative to the nasal bridge; separable components can be provided that are positioned between the lateral wall of the nasal cavity and the nasal septum into which the device docks; the insertion member can be configured to transmit imaging information and/or nasal anatomy information to an operator. The insertion member of the devices can also be configured to expand a space between a nasal septum and a lateral wall of the nasal cavity.

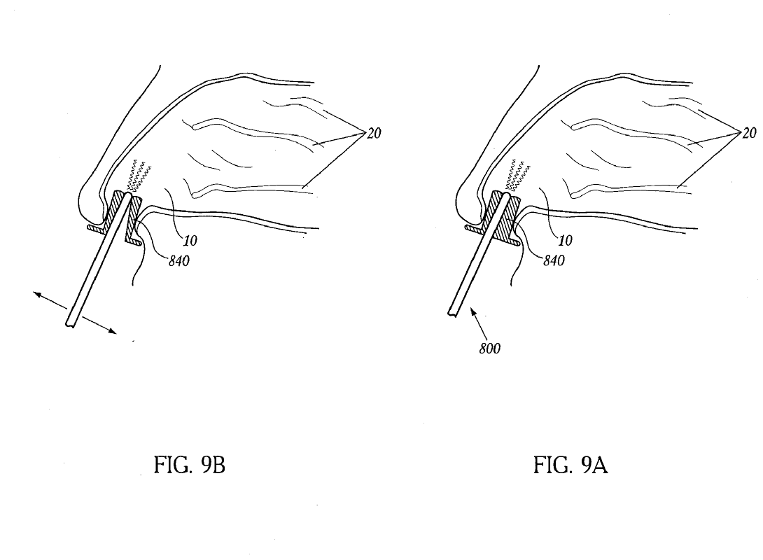

Turning now to FIG. 9, yet another therapy device 800 is depicted wherein the device is placed relative to the nasal cavity 10. In one embodiment, the device 800 is configured to provide an insertion member and a light source that is adapted to communicate with a distal end of the insertion member to illuminate target tissue within a nasal cavity posterior a limen nasi of the nasal cavity, such as respiratory epithelium, and to prevent illumination of non-target tissue, such as squamous epithelium. In another embodiment, the device 800 can be configured to provide one or more light sources and an insertion member that delivers therapeutic light to a target area of the nasal cavity while preventing light delivery to a non-target area without visualization of the nasal cavity by an operator. Still another embodiment, provides an insertion member having sensors adapted to provide nasal structure feedback to an operator and one or more light sources adapted to illuminate the nasal cavity.

FIGS. 9A-B illustrate a device 800 used in combination with a cone 840. The cone 840 is configured so that it engages a distal portion of the device; in some embodiments, the cone is entirely separable from the distal portion of the device. In some embodiments, the cone is removable and/or reusable. The cone can be configured as illustrated in FIG. 9A such that the cone snugly engages the distal end of the device and then snugly fits within the nasal cavity. In this embodiment, the cone 840 directs the illumination of the light therapy device to a target region in the nasal cavity 10. Alternatively, the cone can be configured as illustrated in FIG. 9B such that the cone 840 snugly engages a portion of the distal end of the device while allowing a proximal area to move (illustrated by arrows) within the proximal end of the tip. In this embodiment, the cone 840 allows the light therapy device to be manipulated. The cone 840 may be dimensioned to constrain the limits of manipulation in order to direct the illumination to specific target region(s). In some embodiments, the cone 840 is engaged with the device prior to insertion into the nasal cavity; whereas, in other embodiments, the cone 840 is first placed in the nasal cavity, then the device is docked into the cone 840. The cone 840 can also serve to protect the tissue of the nasal cavity from mechanical abrasion resulting from manipulation of the distal end of the device. The cone 840 may also be dimensioned to position the distal end of the device to illuminate target regions posterior to the limen nasi. Additionally, the cone can be dimensioned to prevent illumination of other regions of the nasal cavity. The external surface of the cone 840 is typically made of a soft material such as rubber, elastomer or polymer.

Similar to other embodiments, it will be appreciated by those skilled in the art, upon review of the disclosure, other features can be incorporated into the design of the embodiments of FIG. 9, as desired. For example, a depth gauge, or ruler, can be provided to assist in determining the depth of light delivery; the device can be adapted to be positionable relative to the nasal bridge; separable components can be provided that are positioned between the lateral wall of the nasal cavity and the nasal septum into which the device docks; the insertion member can be configured to transmit imaging information and/or nasal anatomy information to an operator. The insertion member of the devices can also be configured to expand a space between a nasal septum and a lateral wall of the nasal cavity.

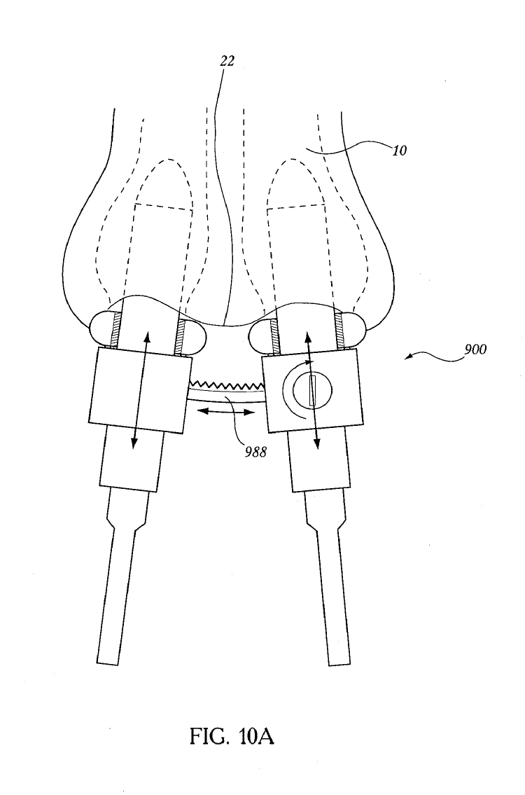

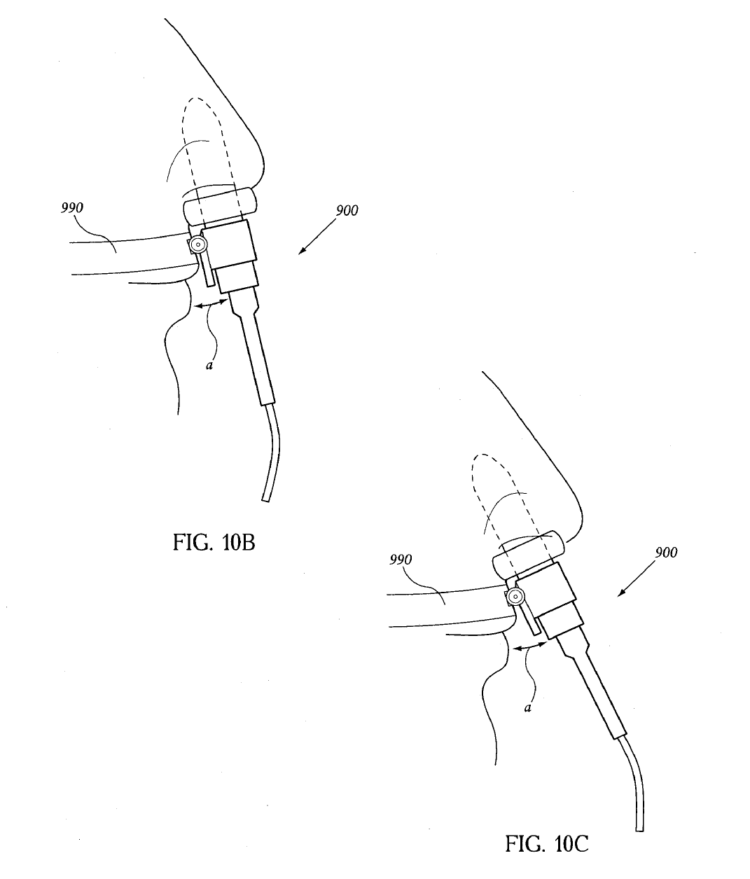

Turning now to FIG. 10, yet another therapy device 900 is depicted wherein the device is placed relative to the nasal cavity 10. An optical therapy device for providing therapeutic light to a nasal cavity, comprising: an insertion member configured to pivot relative to a connector positioned external a nasal cavity of a patient; and one or more light sources adapted to illuminate the nasal cavity. In one embodiment, the device is configured to provide an insertion member and a light source that is adapted to communicate with a distal end of the insertion member to illuminate target tissue within a nasal cavity posterior a limen nasi of the nasal cavity, such as respiratory epithelium, and to prevent illumination of non-target tissue, such as squamous epithelium. In another embodiment, the device can be configured to provide one or more light sources and an insertion member that delivers therapeutic light to a target area of the nasal cavity while preventing light delivery to a non-target area without visualization of the nasal cavity by an operator. Still another embodiment provides an insertion member having sensors adapted to provide nasal structure feedback to an operator and one or more light sources adapted to illuminate the nasal cavity.

In FIGS. 10A-C, a device 900 is depicted that is positioned and left in place during the phototherapy treatment. The device of FIG. 10 is configured to treat both nasal cavities at one time and is provided with a bridge or connector 988 between the two nasal treatment devices and which is positioned on the nasal bridge 22. In FIG. 10A, the connector 988 can adjust the relative distance or angle between the two nasal treatment devices. This can be accomplished through a slideable or pivoting mechanism. As shown in the side-view depicted in FIG. 10B-C it may be desirable to secure the device 900 using, for example, a securement 990 to the patient so that the device remains fixed in place. As illustrated, a mechanism for securing the device by providing an adjustable loop that fits around a patient's head is provided. The loop could be elastic or rigid. Other configurations that enable the device to be secured include, for example, attachment devices to connect to a patient's eyewear, a strap or loop around the ears, a strap around the head, a bite piece, adhesive tape, as well as other suitable mechanisms. Alternatively, the securement is provided by a mechanical fixture not attached to the patient such as those used to stabilize the head and eyes in ophthalmology. Additionally, the angle a between the patient's face and the orientation of the device can be adjusted as desired, by rotating or pivoting the device 900 relative to the securement 990. Similar to other embodiments, it will be appreciated by those skilled in the art, upon review of the disclosure, other features can be incorporated into the design of the embodiments of FIG. 10, as desired. For example, a depth gauge, or ruler, can be provided to assist in determining the depth of light delivery; the device can be adapted to be positionable relative to the nasal bridge; separable components can be provided that are positioned between the lateral wall of the nasal cavity and the nasal septum into which the device docks; the insertion member can be configured to transmit imaging information and/or nasal anatomy information to an operator. The insertion member of the devices can also be configured to expand a space between a nasal septum and a lateral wall of the nasal cavity.

Turning now to FIG. 11, yet another therapy device 1000 is depicted wherein the device is placed relative to the nasal cavity 10. In one embodiment, the device is configured to provide an insertion member and a light source that is adapted to communicate with a distal end of the insertion member to illuminate target tissue within a nasal cavity posterior a limen nasi of the nasal cavity, such as respiratory epithelium, and to prevent illumination of non-target tissue, such as squamous epithelium. In another embodiment, the device can be configured to provide one or more light sources and an insertion member that delivers therapeutic light to a target area of the nasal cavity while preventing light delivery to a non-target area without visualization of the nasal cavity by an operator. Still another embodiment provides an insertion member having sensors adapted to provide nasal structure feedback to an operator and one or more light sources adapted to illuminate the nasal cavity.

In FIG. 11A, a device 1000 is depicted that is positioned and left in place during the phototherapy treatment. Exterior bars 1091 that engage markers 1092 are provided that enable the user to position the device 1000 within the nasal cavity 10 by positioning the markers 1092 relative to an exterior marking on the nose that corresponds to the nasal valve and limen nasi within the interior of the nose. One such exterior marking could be the nasal alar. Where the device is adapted to treat both nasal cavities at once, a bridge or connector can connect the two treatment devices and facilitate positioning the devices within the nasal cavity. Similar to the device shown in FIG. 10B a securement can be provided so that the device remains fixed in place.

In FIG. 11B, the device 1000 is configured to have a second insertion member 1022 that engages the first insertion member 1021. The second insertion member 1022 can be positioned to adjust the depth of the first insertion member 1021. The design of this embodiment accommodates a range of patient nose sizes and depths, including the depth of the nasal valve from the nasal bridge. In one embodiment, a single use sheath 1018 is provided and is designed to enable a the re-positioning of the first insertion member 1021 with the second insertion member 1022. The single use sheath 1018 can be of at least partially of a flexible thin wall construction which allows the re-positioning without creating a gap or opening in the sheath. In this manner, the cleanliness of the insertion members is maintained. A slidable mechanism 1024 is provided to enable the device to adjust the depth of the two nasal treatment components 1021, 1021′ in the nasal cavity 1020, 1020′. The sheath 1018 can be adapted to fit over an optical handle assembly 1000 and has a middle section that registers in a patient specific manner to a patient's nasal bridge 22; or can be formed integrally with the light guide 1000.

The sheath 1018 covers all surfaces that contact the patient, thus eliminating the need to clean, disinfect or sterilize the device between patients. The sheath material can be comprised of any suitable biocompatible material, including elastomers. The design allows one tip to accommodate a range of patient sizes. A bridge adjustor 1024 is provided that is adapted to enable the device to adjust the depth of the nasal treatment components 1021, 1021′ by sliding the device up-and-down s relative to the nasal bridge 22. Light 1001 is delivered distally through the device.

As will be appreciated by those skilled in the art, there are other embodiments where a second insertion member engages a first insertion member. For example, the first insertion member may only provide light 1001 to one nasal cavity. In another embodiment, the first insertion member may have notches, holes, or other patterns to allow the engaging of the second insertion member. Alternatively, the second insertion member may be partially or fully constructed onto the first insertion member. For example, the second insertion member may be a pin, lever, or other component which extends from the first insertion member.

Similar to other embodiments, it will be appreciated by those skilled in the art, upon review of the disclosure, other features can be incorporated into the design of the embodiments of FIG. 11, as desired. For example, a depth gauge, or ruler, can be provided to assist in determining the depth of light delivery; the device can be adapted to be positionable relative to the nasal bridge; separable components can be provided that are positioned between the lateral wall of the nasal cavity and the nasal septum into which the device docks; the insertion member can be configured to transmit imaging information and/or nasal anatomy information to an operator. The insertion member of the devices can also be configured to expand a space between a nasal septum and a lateral wall of the nasal cavity.

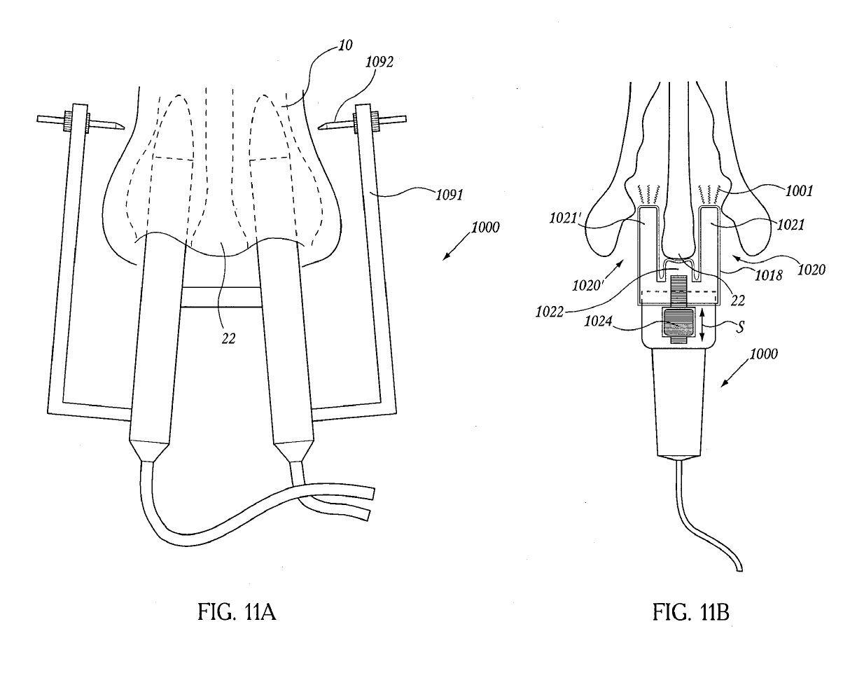

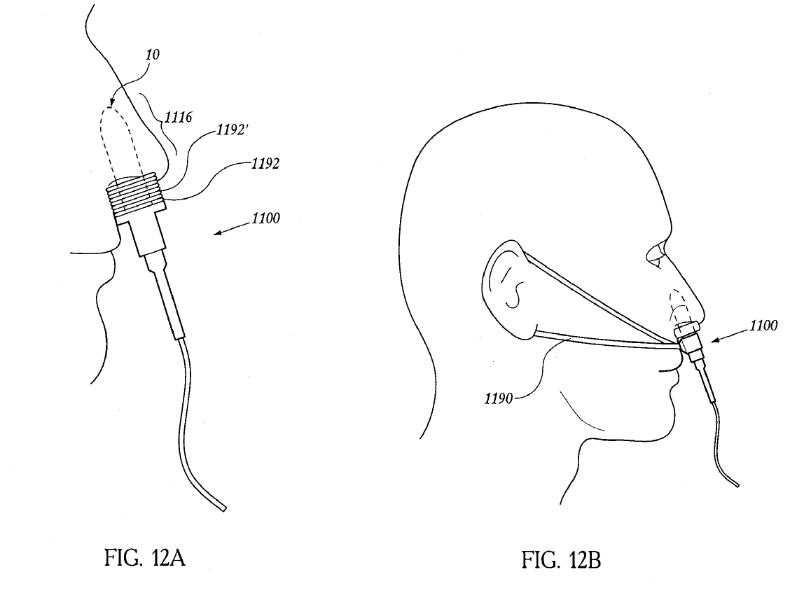

Turning now to FIG. 12, yet another therapy device 1100 is depicted wherein the device is placed relative to the nasal cavity 10. In one embodiment, the device is configured to provide an insertion member and a light source that is adapted to communicate with a distal end of the insertion member to illuminate target tissue within a nasal cavity posterior a limen nasi of the nasal cavity, such as respiratory epithelium, and to prevent illumination of non-target tissue, such as squamous epithelium. In another embodiment, the device can be configured to provide one or more light sources and an insertion member that delivers therapeutic light to a target area of the nasal cavity while preventing light delivery to a non-target area without visualization of the nasal cavity by an operator. Still another embodiment, provides an insertion member having sensors adapted to provide nasal structure feedback to an operator and one or more light sources adapted to illuminate the nasal cavity.

FIG. 12A illustrates a device 1100 which has one or more nose pieces that are adapted to provide an adjustable depth of the distal tip by providing one or more washers or spacers 1192, 1192′. Addition of washers or spacers 1192 shortens or lengthens the depth of the distal tip 1116 within the nasal cavity 10; removal of washers or spacers increases the depth of the distal tip. Additionally, the washers or spacers 1192 can be configured such that each one has a height that is either fixed (e.g., as in the case of a spacer formed from metal), or adaptable (e.g., where the spacer is formed from, for example, an elastomeric material). Where the spacer has an adaptable height, the spacers can be compressed together to vary the amount of distal tip available for insertion into the nasal cavity. Additionally, the spacers 1192 can be of variable thickness. Alternatively, a single spacer can be positioned and secured onto the nose piece to vary the depth of the distal tip.

The device 1100 can be provided with a means of securement 1190 as shown in FIG. 12B and as described above. As will be appreciated by those skilled in the art, the securement 1190 can be incorporated into any of the embodiments illustrated in this application, including, for example, FIGS. 4-11 and 14-15.

Similar to other embodiments, it will be appreciated by those skilled in the art, upon review of the disclosure, other features can be incorporated into the design of the embodiments of FIG. 12, as desired. For example, a depth gauge, or ruler, can be provided to assist in determining the depth of light delivery; the device can be adapted to be positionable relative to the nasal bridge; separable components can be provided that are positioned between the lateral wall of the nasal cavity and the nasal septum into which the device docks; the insertion member can be configured to transmit imaging information and/or nasal anatomy information to an operator. The insertion member of the devices can also be configured to expand a space between a nasal septum and a lateral wall of the nasal cavity.

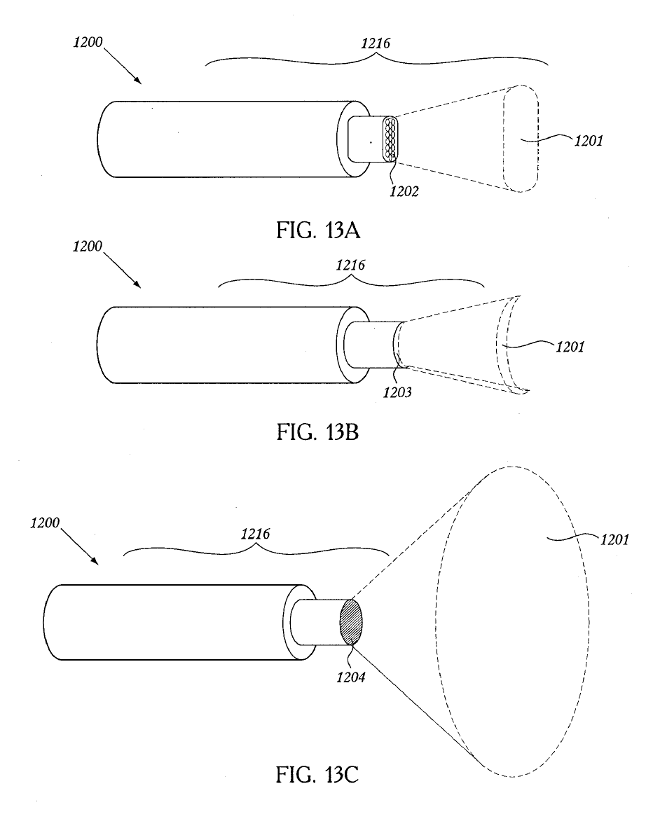

Turning now to FIG. 13, illustrates distal ends for use in therapy devices that are placed relative to the nasal cavity 10. In one embodiment, the distal end depicted is adapted for use in a device that is configured to provide an insertion member and a light source that is adapted to communicate with a distal end of the insertion member to illuminate target tissue within a nasal cavity posterior a limen nasi of the nasal cavity, such as respiratory epithelium, and to prevent illumination of non-target tissue, such as squamous epithelium. In another embodiment, the distal end depicted is adapted for use in a device that can be configured to provide one or more light sources and an insertion member that delivers therapeutic light to a target area of the nasal cavity while preventing light delivery to a non-target area without visualization of the nasal cavity by an operator. Still another embodiment, the distal end configured is adapted to be used in a device that provides an insertion member having sensors adapted to provide nasal structure feedback to an operator and one or more light sources adapted to illuminate the nasal cavity.

As shown in FIGS. 13A-C, the device 1200 is adapted at the distal end 1216 to accommodate a nasal cavity having a non-uniform surface. In order to optimize light distribution, the light 1201 emitted from the distal end 1216 can be conditioned. In one embodiment, the device can be adapted to create a non-circular illumination beam as the nasal cavity is narrower than wide. As shown in FIG. 13A, fiber optics 1202 can be arranged in a pattern, while FIG. 13B illustrates the use of a lens 1203 with custom optics. Another option is to diffuse the light as illustrated in FIG. 13c by spreading the light out a\with a diffusing component 1204. This can be accomplished by passing light through a diffusing material such as PTFE, creating surface roughness on the surface of the distal end, or the like.

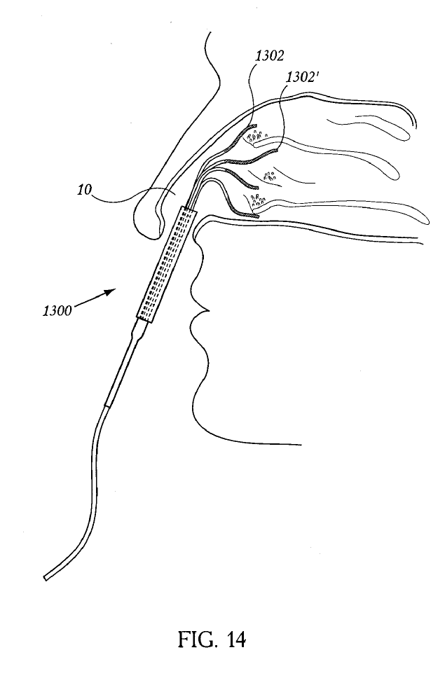

Turning now to FIG. 14, yet another therapy device 1300 is depicted wherein the device is placed relative to the nasal cavity 10. In one embodiment, the device is configured to provide an insertion member and a light source that is adapted to communicate with a distal end of the insertion member to illuminate target tissue within a nasal cavity posterior a limen nasi of the nasal cavity, such as respiratory epithelium, and to prevent illumination of non-target tissue, such as squamous epithelium. In another embodiment, the device can be configured to provide one or more light sources and an insertion member that delivers therapeutic light to a target area of the nasal cavity while preventing light delivery to a non-target area without visualization of the nasal cavity by an operator. Still another embodiment, provides an insertion member having sensors adapted to provide nasal structure feedback to an operator and one or more light sources adapted to illuminate the nasal cavity.

With respect to FIG. 14, another device 1300 having the characteristics described below, is provided that is adapted for distributing light within the nasal cavity 10 by providing protrusions which carry light to different areas of the nasal cavity. The protrusions 1302 could be rigid or flexible; examples of flexible protrusions include soft elastomers or UV transmissive silicone rubber. Where light is distributed within the cavity, it will be appreciated that each of the light distributing elements 1302, 1302′ can be configured to deliver a different wavelength or intensity of light. Additionally, each of the light distributing elements 1302, 1302′ can be configured to target specific regions of the nasal cavity and hence may be geometrically different in length, shape, thickness, etc. Light may exit the side and/or the end of the protrusion. The light exit region of the protrusion 1302 may be configured to optimize light distribution, including but not limited to custom a lens shape or diffusing properties. Additionally, the protrusions 1302 may also contain light scattering particles, as described above, to optimize distribution of the therapeutic light. The protrusions may be custom moldable to uniquely fit a patient's anatomy, or have preformed shapes for specific target regions, or adapted to fit a variety of nasal cavity anatomies.

Similar to other embodiments, it will be appreciated by those skilled in the art, upon review of the disclosure, other features can be incorporated into the design of the embodiments of FIG. 14, as desired. For example, a depth gauge, or ruler, can be provided to assist in determining the depth of light delivery; the device can be adapted to be positionable relative to the nasal bridge; separable components can be provided that are positioned between the lateral wall of the nasal cavity and the nasal septum into which the device docks; the insertion member can be configured to transmit imaging information and/or nasal anatomy information to an operator. The insertion member of the devices can also be configured to expand a space between a nasal septum and a lateral wall of the nasal cavity.

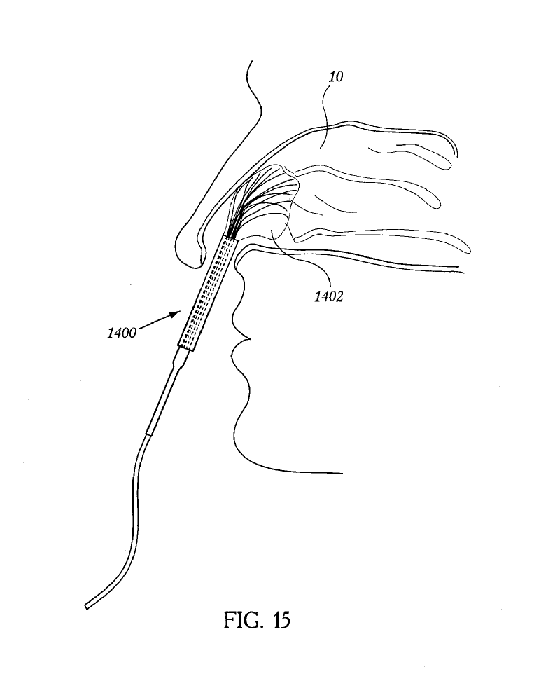

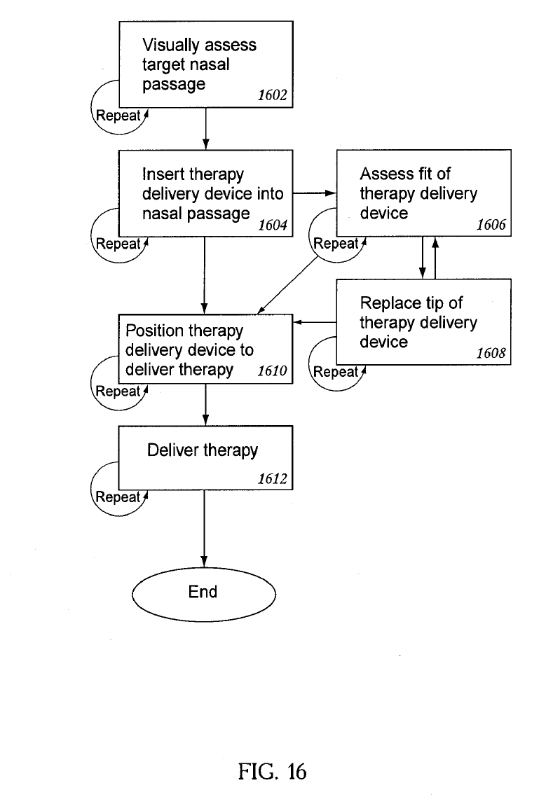

Turning now to FIG. 15, yet another therapy device 1400 is depicted wherein the device is placed relative to the nasal cavity 10. In one embodiment, the device is configured to provide an insertion member and a light source that is adapted to communicate with a distal end of the insertion member to illuminate target tissue within a nasal cavity posterior a limen nasi of the nasal cavity, such as respiratory epithelium, and to prevent illumination of non-target tissue, such as squamous epithelium. In another embodiment, the device can be configured to provide one or more light sources and an insertion member that delivers therapeutic light to a target area of the nasal cavity while preventing light delivery to a non-target area without visualization of the nasal cavity by an operator. Still another embodiment, provides an insertion member having sensors adapted to provide nasal structure feedback to an operator and one or more light sources adapted to illuminate the nasal cavity.

Turning to FIG. 15, another device 1400 having the characteristics described below is illustrated for distributing light within the nasal cavity 10. In this embodiment, an expanding element 1402 that spreads the optics to fit the patient's anatomy is provided. Such an element can be an elastomer or foam. The expanding element could also be configured to transmit light, as in a UV transmissive silicone rubber, such as GE RTV-615 or Dow Coming Sylgard 184 or EG-6301. Additionally, the device can be adapted to contain light transmissive elements such as UV transmitting quartz silica fibers. By forming to a patient's unique anatomy, therapeutic light can be more optimally delivered.

Similar to other embodiments, it will be appreciated by those skilled in the art, upon review of the disclosure, other features can be incorporated into the design of the embodiments of FIG. 15, as desired. For example, a depth gauge, or ruler, can be provided to assist in determining the depth of light delivery; the device can be adapted to be positionable relative to the nasal bridge; separable components can be provided that are positioned between the lateral wall of the nasal cavity and the nasal septum into which the device docks; the insertion member can be configured to transmit imaging information and/or nasal anatomy information to an operator. The insertion member of the devices can also be configured to expand a space between a nasal septum and a lateral wall of the nasal cavity.

As will be appreciated by those skilled in the art, a variety of methods can be employed for applying phototherapy to a selected region of a nasal cavity. For example, the method can comprise: (a) inserting an insertion member adapted to selectively deliver a light emitter into the nasal cavity; (b) positioning the insertion member at or near a target region; and (c) delivering phototherapy to a target region of the nasal cavity. As illustrated in FIG, 16 an operator, such as a physician, visually assesses the target nasal passage 1602, this step can be repeated as often as required or desired. Once the physician determines which nasal passage is a target for treatment, a therapy device is inserted into the nasal passage 1604. The device can be any of the devices disclosed herein. Optionally, the fit of the therapy device can be assessed 1606, one or more times. Additionally, the user can optionally replace the tip 1608 of the therapy device. Replacing the tip might be desirable where the tips are configured to accommodate anatomical differences of a patient. After the therapy device is delivered 1604, or following the any of the optional steps, the position of the therapy delivery device is determined without visualization of the distal tip of the therapy device 1610. This step can be achieved by a variety of methods based on the devices disclosed above. Once the position of the therapy delivery device is positioned distal to the limen nasi, therapy is delivered 1612. A variety of additional steps can be included depending on the device selected. For example, the limen can be wedged open. Additional steps can also include treating a first and second nasal cavity, adjusting a depth of a distal tip of the insertion member, adjusting a distance between a base of a first insertion member and a base of a second insertion member, and/or adjusting an angle between an axis of a first insertion member and an axis of a second insertion member. In some embodiments of the method, the method can also comprise the step of positioning an external depth gauge adjacent a target anatomical location, positioning a removable insertion guide over the insertion member prior to inserting the insertion member into the nasal cavity, and/or securing the insertion member to a patient. In still other embodiments, the method can include the step of condition the phototherapy prior to delivery to the target region of the nasal cavity. It can also be advantageous, in some embodiments, to measure the anatomic dimensions of the nasal cavity.

Another embodiment of the invention includes a method of treating a selected region of a nasal cavity. The method comprises: (a) inserting an insertion member adapted to selectively deliver a light emitter into the nasal cavity; (b) positioning the insertion member either in the vestibule of the nose or distal to the vestibule of the nasal cavity without visualization of the distal end of the insertion member by an operator; and (c) delivering phototherapy to a target region of the nasal cavity. The method can also include the step of wedging open the limen. Additional steps can also include treating a first and second nasal cavity, adjusting a depth of a distal tip of the insertion member, adjusting a distance between a base of a first insertion member and a base of a second insertion member, and/or adjusting an angle between an axis of a first insertion member and an axis of a second insertion member. In some embodiments of the method, the method can also comprise the step of positioning an external depth gauge adjacent a target anatomical location, positioning a removable insertion guide over the insertion member prior to inserting the insertion member into the nasal cavity, and/or securing the insertion member to a patient. In still other embodiments, the method can include the step of condition the phototherapy prior to delivery to the target region of the nasal cavity. It can also be advantageous, in some embodiments, to measure the anatomic dimensions of the nasal cavity. In further methods, a cream such as a sunscreen or other UV blocking cream can be applied to the vestibule prior to application of the light so as to prevent light from reaching unwanted regions of the nasal cavity.

### CLINICAL EXAMPLES

In the examples that follow, clinical scenarios are presented which illustrate the discoveries and inventions described herein.

### CLINICAL EXAMPLE 1

Three minutes of rhinophototherapy was applied to the nostril of a patient on consecutive days (days 1 and 2) using a rhinotherapy tip and similar spectrum as is taught by Kemeny et. al. in patent application Ser. No. 10/440,690. Examination by speculum and nasal endoscopy prior to the first therapy revealed normal appearing nasal cavity including the transition zone (limen nasi) region and the inferior turbinate. By day 4, “dryness” was encountered by the patient. Repeat examination of the nasal cavity on day 4 revealed a hyperproliferative response at the region of the vestibular limen manifest as a “crusting.” The inferior turbinate region and remainder of the nasal respiratory mucosa continue to appear normal.

### CLINICAL EXAMPLE 2

In a subsequent experiment in the same patient, the same rhinophototherapy dose was applied through a tip with a balloon placed around the outside with the rhinophototherapy tip within the balloon. Ten milliliters of saline was placed in the balloon after the balloon was inserted in the nasal cavity at the region of the vestibular limen. The therapy device was then pivotable around the nasal cavity easily and without any discomfort to the patient. Two treatments were applied on sequential days. The patient did not complain of dryness on day 4 or any subsequent day. Endoscopy of the vestibular region of the nasal cavity did not reveal any crusting or other abnormalities of the nasal cavity.

### CLINICAL EXAMPLE 3

Three minutes of rhinophototherapy was applied to the nostril of a patient on consecutive days (days 1 and 2) using a tip adapted and configured to control depth. This tip has a depth gauge and a stop attached to it which prevents movement past the prescribed depth and also importantly prescribes a minimum depth to keep the device in order to position it past limen region and transition zone. The depth of the depth gauge is adjusted based on a distance of the vestibular limen from the entrance to the nostril; one example of the interface for the depth gauge is the bridge of the nostril. The depth gauge provides a haptic indicator of where the device should be placed; otherwise, as in the example 1, the device can be applied too deeply or too shallow in the nose of the patient and the operator cannot judge where to place the applicator while applying the therapy. In this example, phototherapy was applied to the nose of a patient for three minutes on consecutive days (days 1 and 2). The patient did not complain of symptoms of dryness or irritation and there were no hyperproliferative lesions seen in the vestibular region.

### CLINICAL EXAMPLE 4

This example follows a similar protocol to examples 1-3. However, in this example, a sunscreen was applied to the nasal limen region of the nasal cavity of the patient just prior to treatment with the rhinophototherapy device. In this example, despite several treatments of rhinophototherapy, the volunteer did not experience “dryness.” Because light to the region anterior to the nasal limen was blocked by the sunblock, this example offers further proof that irritation of the squamous epithelium can be avoided by preventing light exposure to the region.

As can be seen from these device and clinical examples, a preferred method of applying phototherapy to the nasal cavity is to avoid light exposure of the region of squamous cell epithelial cells anterior to the vestibular limen. It is particularly advantageous to apply nasal phototherapy with a technique that does not involve visualization of the internal nasal anatomy and therefore devices which enable this technique must be designed with knowledge of the nasal anatomy and the effects of light on specific parts of the nasal anatomy.

A further appreciation of the embodiments of this invention can be appreciated from the disclosures of U.S. patent application Ser. No. 11/276,787 filed Mar. 14, 2006 to Gertner et al entitled Devices, Methods and Kits for Radiation Treatment Via a Target Body Surface; Ser. No. 11/244,812, filed Oct. 5, 2005 to Gertner et al. entitled Devices, Methods and Kits for Radiation Treatment Via a Target Body Surface; application Ser. No. 11/152,946 filed Jun. 14, 2005 by Gertner et al. for Optical Therapies and Devices; application Ser. No. 11/340,824 filed Jan. 25, 2006 by Gertner et al. for Optical Therapies Device for Providing Therapy to a Body Cavity; as well as underlying U.S. Provisional Application Nos. 60/690,792, filed Jun. 15, 2005; 60/661,688 filed Mar. 14, 2005; and 60/646,818 filed Jan. 25, 2005.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. An optical therapy device for providing therapeutic light to a target region of tissue within a nasal cavity, comprising:
a first insertion member;
a light source communicating with a distal end of the first insertion member, wherein light from the light source illuminates a target region of tissue within a nasal cavity posterior a to limen nasi of the nasal cavity, and further wherein the insertion member is configured to prevent illumination of a region of tissue anterior to the limen nasi.

2. The device of claim 1 wherein the insertion member comprises a light conditioner configured to condition light exiting the distal end.

3. The device of claim 2 wherein the light conditioner is selected from the group consisting of: light scattering particles, a lens, a mirror, a filter, and a diffuser.

4. The device of claim 1 wherein the insertion member comprises an expandable component configured for positioning in a space between a nasal septum and a lateral wall of the nasal cavity.

5. The device of claim 4 wherein the expandable component transmits light.

6. The device of claim 1 wherein at least a portion of the insertion member comprises a soft material.

7. The device of claim 6 wherein the soft material is selected from the group consisting of: an elastomer, a rubber, a polymer, a hydrogel, a viscoelastic material, a hydrophilic material, and a hydrophobic material.

8. The device of claim 1 wherein the insertion member is further configured to provide a nasal anatomy information to an operator.

9. The device of claim 1 wherein the insertion member is further configured to transmit imaging information to an operator.

10. The device of claim 1 wherein the insertion member further comprises a depth gauge to set a depth of light delivery.

11. The device of claim 1 wherein the insertion member comprises a ruler.

12. The device of claim 1 wherein the insertion member comprises a first insertion member, the optical therapy device further comprising a second insertion member engaging the first insertion member.

13. The device of claim 12 wherein the second insertion member is configured to adjust the depth of the first insertion member.

14. The device of claim 12 wherein the second insertion member is positionable relative to a nasal bridge.

15. The device of claim 1 wherein the insertion member comprises a separable component adapted and configured to be positioned between a lateral wall of a nasal cavity and the nasal septum and a second component which fits onto the optical therapy device and which is adapted to dock in the separable component.

16. The device of claim 15 where the separable component is configured to position a depth of the second component within the nasal cavity.

17. The device of claim 15 wherein the separable component is configured to determine an angle at which the second component directs the optical therapy device.

18. The device of claim 1 wherein the insertion member has one or more stackable elements configured to position a depth of the distal end within the nasal cavity.

19. The device of claim 1 further comprising a securement device adapted to engage a patient.

20. The device of claim 19 wherein the securement device is adapted to position the insertion member in the nasal cavity.

21. An optical therapy device for providing therapeutic light to a nasal cavity, comprising:
one or more light sources; and
an insertion member adapted to deliver therapeutic light from a light source to a first region of the nasal cavity while preventing light delivery to a second region, wherein the insertion member is further adapted to be positioned within the nasal cavity without visualization of the nasal cavity by an operator.

22. The optical therapy device of claim 21 wherein the first region comprises respiratory epithelium and the second region is the squamous epithelium of the nasal vestibule.

23. The optical therapy device of claim 21 wherein the second region is one or more regions selected from the group consisting of a nasal vestibule, nasal septum, and a nasal valve.

24. The optical therapy device of claim 21 wherein the insertion member comprises a light conditioner further configured to condition light exiting a light exit region.

25. The optical therapy device of claim 21 wherein the insertion member further comprises an expandable component adapted to expand a space between a nasal septum and a lateral wall of the nasal cavity.

26. The optical therapy device of claim 21 wherein the insertion member is further adapted to partially block light from the light emitters.

27. The optical therapy device of claim 21 wherein the insertion member comprises of a first part adapted and configured to expand a region between a lateral wall of a nasal cavity and the nasal septum and a second part having an optical therapy device adapted to dock in the first part.

28. An optical therapy device for providing therapeutic light to a nasal cavity, comprising:
an insertion member having a sensor adapted to provide nasal structure feedback to an operator; and
one or more light sources adapted to illuminate the nasal cavity.

29. The optical therapy device of claim 28 wherein the feedback is visual information from an imaging element associated with the device.

30. The optical therapy device of claim 29 wherein the visual information is obtained by way of a semiconductor imaging element.

31. A method of applying phototherapy to a target region of a nasal cavity comprising: (a) inserting a device having an insertion member into the nasal cavity; (b) positioning the insertion member at or near a target region; and (c) selectively delivering phototherapy to the target region of the nasal cavity.

32. The method of claim 31 wherein the insertion member is adapted to transmit target region information to an operator of the device regarding the target regions of the nasal cavity.

33. The method of claim 32 wherein the target region information is visual.

34. The method of claim 32 wherein the target region information is anatomic measurements.

35. The method of claim 31 further comprising the step of wedging open the limen nasi.

36. The method of claim 31 further comprising adjusting a depth at which the distal tip of the insertion member emits light.

37. The method of claim 31 wherein the step of inserting a device further comprises the step of adjusting a distance between a base of a first insertion member and a base of a second insertion member.

38. The method of claim 31 wherein the step of inserting a device further comprises the step of adjusting an angle between an axis of a first insertion member and an axis of a second insertion member.

39. The method of claim 31 wherein the step of inserting a device further comprises the step of positioning an external depth gauge adjacent a target anatomical location.

40. The method of claim 31 wherein the step of inserting a device further comprises the step of positioning a removable insertion guide over the insertion member prior to inserting the insertion member into the nasal cavity.

41. The method of claim 31 wherein the step of inserting a device further comprises the step of positioning a cap in the nasal cavity prior to a step of docking the insertion member into the cap.

42. The method of claim 31 further comprising the step of securing the insertion member to a patient.

43. The method of claim 31 further comprising the step of conditioning the phototherapy prior to delivery to the target region of the nasal cavity.

44. The method of claim 31 further comprising the step of measuring an anatomy of the nasal cavity.

45. An optical therapy device for providing therapeutic light to a nasal cavity, comprising:
an insertion member configured to pivot relative to a connector positioned external a nasal cavity of a patient; and
one or more light sources adapted to illuminate the nasal cavity.

46. The optical therapy device of claim 45 comprising a second insertion member configured to pivot relative to the connector.

47. The optical therapy device of claim 46 further comprising a bridge between the first and second insertion members.

48. The optical therapy device of claim 47 wherein the first or second insertion member slideably engages the bridge.

49. The optical therapy device of claim 45 wherein the insertion member pivots a distal end of the insertion member relative to the connector.
